(19) United States
(12) Patent Application Publication
(10) Pub. No.: US 2016/0311764 A1
(43) Pub. Date: Oct. 27, 2016

(54) ACTIVATORS OF MYOSIN II FOR MODULATING CELL MECHANICS

(71) Applicant: THE JOHNS HOPKINS UNIVERSITY, Baltimore, MD (US)

(72) Inventors: Douglas Robinson, Towson, MD (US); Alexandra Surcel, Baltimore, MD (US)

(21) Appl. No.: 15/103,665

(22) PCT Filed: Dec. 16, 2014
(86) PCT No.: PCT/US2014/070619

§ 371 Date: Jun. 10, 2016

Related U.S. Application Data
(60) Provisional application No. 61/916,404, filed on Dec. 16, 2013.

(51) Int. Cl. C07C 271/58; C12Q 1/02; C07C 49/825; C07C 251/80; A61K 31/12; A61K 31/136

(52) U.S. Cl. CPC C07C 271/58; A61K 31/12; A61K 31/136; C07C 49/825; C07C 251/80; C12Q 1/025; G01N 2500/10; G01N 2333/44

(57) ABSTRACT

The present invention discloses small molecule compounds as activators of myosin II by promoting its assembly and recruitment to contractile structures in the cell and methods of using such compounds. These compounds are useful to modulate cell and tissue mechanics. This class of molecules, which affect cell mechanics either by activating the contractile system of the cell or modulating cytokinesis, will be used for therapeutic and tissue engineering applications.

### CROSS-REFERENCE TO RELATED APPLICATIONS

This application claims benefit from U.S. Provisional Application 61/916,404, filed Dec. 16, 2013. This application is incorporated herein by reference for all purposes.

### STATEMENT REGARDING FEDERALLY SPONSORED RESEARCH

This invention was made with government support under GM66817 awarded by National Institutes of Health. The government has certain rights in the invention.

### BACKGROUND OF THE INVENTION

The present invention relates generally to compounds as activators of myosin II by promoting its assembly and recruitment to contractile structures in the cell and methods of using such compounds. These compounds may be used to modulate cell and tissue mechanics. This class of molecules, which activate the contractile system of the cell, may also be used for therapeutic and tissue engineering applications.

In the U.S., one in two people will develop cancer and one in three will acquire cardiovascular disease during their lifetime. These conditions depend on contractile systems driving the cell mechanics of division, mechanosensing, motility or cardiomyocyte contraction. Consequently, each may be impaired by molecules that modulate the cell's mechanical machinery. Cell mechanics are central to healthy and pathological states of cells, tissues and organ formation and function.

There are known major classes of myosin II modulating compounds. For example, Omecamtiv mecarbil (Cytokinetics, INC.) (Malik, Hartman, et al., 2011) is an activator of the catalytic activity of the myosin II motor by promoting tight binding to actin filaments and is specific for cardiac myosin II. Blebbistatin is an inhibitor of the myosin II motor domain and works by blocking phosphate release (Straight, Cheung, et al., 2003).

There are other known compounds that inhibit myosin II activity. For example, BDM inhibits the ATPase activity of skeletal myosin II (e.g., Ostap, 2002). Calyculin A targets PP1- and PP2A-type protein phosphatases and leads to increased myosin II activity (e.g., Ishihara, Martin, et al., 1989; Ishihara, Ozaki, et al., 1989). Myosin light chain phosphorylation inhibitors include myosin light chain kinase (MLCK) inhibitors, such as ML-7 (e.g. Makishima, Honma, et al., 1991; Saitoh, Ishikawa, et al., 1987), and Rho kinase (ROCK) inhibitors, such as Y-27632 (e.g., Uehata, Ishizaki, et al., 1997); these compounds reduce myosin activation.

Nevertheless, it would be desirable to identify small molecules for directly promoting myosin II accumulation and recruitment to contractile structures.

### SUMMARY OF THE INVENTION

The present invention overcomes the aforementioned drawbacks by providing small molecules as myosin II activators for promoting myosin II accumulation and recruitment to contractile structures where cell tension and elasticity is increased.

In one embodiment, the present invention discloses a method for modulating cell mechanics of a disease condition in a subject comprising the step of administering an effective amount of a compound (I) or its derivatives, or a combination of their constituents, wherein the compound (I) has the formula:

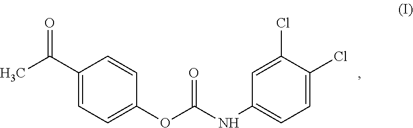 [SMILES: CC(=O)c1ccc(OC(=O)Nc2ccc(Cl)c(Cl)c2)cc1]

wherein myosin II is activated, cell mechanics are modulated and the disease condition is treated in the subject.

In one embodiment, the present invention discloses a method for modulating cell mechanics of a disease condition in a subject comprising the step of administering an effective amount of a compound (II) or its derivatives, or a combination of their constituents, wherein the compound (II) has the formula:

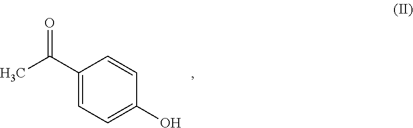

wherein myosin II is activated, cell mechanics are modulated and the disease condition is treated in the subject.

In one embodiment, the present invention discloses a method for modulating cell mechanics of a disease condition in a subject comprising the step of administering an effective amount of a compound (IV) or its derivatives, or a mixture of their constituents, wherein the compound (IV) has the formula:

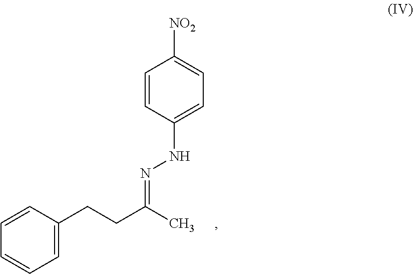 [SMILES: C/C(CCc1ccccc1)=N\Nc1ccc([N+](=O)[O-])cc1]

wherein cytokinesis is modulated and the disease condition is treated in the subject.

In some embodiments, the present invention discloses compounds having formulas of I, II or IV for use in activating myosin II or inhibiting cytokinesis to treat a disease condition in a subject by systemic delivery.

In some embodiments, the present invention discloses pharmaceutical compositions for modulating cell mechanics of a disease condition in a subject comprising a compound having the formulas of I, II or IV. In one embodiment, the pharmaceutical compositions further comprise at least one pharmaceutically-acceptable carrier.

In one aspect, the present invention discloses an in vivo, large-scale and high-throughput screening method for identifying cell mechanical modulators. The screening method comprise the steps of (a) obtaining cells and placing the cells on multiple-well substrate plates for cytokinesis; (b) contacting the cells on multiple-well substrate plates with compound candidates; and (c) monitoring and analyzing the cytokinesis and the growth of the cells.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1(A-D) are a set of diagrams and graphs showing CIMPAQ processes of high-throughput data and identification of mechanical modulators, mitotic inhibitors, and lethal compounds. FIG. 1A shows workflow diagram of primary screening from 384-well plating (i) to raw data acquisition (ii) to CIMPAQ image conversion by segmentation (iii). Cytokinesis hits are identified in a 5-step process: Acquisition of FIG. 1A(ii) raw images of NLS-tdTomato expressing cells and are converted into FIG. 1A(iii) CIMPAQ-processed version. FIG. 1B shows sample histogram of a single well showing the distribution of nuclei per cell counts demonstrating high agreement between manual counts and CIMPAQ analysis. The Cartesian coordinates defined by the ratio of bi- to mono-nucleated cells and the ratio of multi- to mononucleated cells of the untreated WT wells are fitted to a two dimensional Gaussian distribution in FIG. 1C. From this distribution, contour lines for all standard deviations from the control mean are determined for a given plate as shown in FIG. 1D.

FIGS. 2(A-D) are a set of images and graphs showing the molecular structure of carbamate-7 and identification of carbamate-7 as a cytokinesis inhibitor affecting the myosin II-RacE pathway according to one embodiment of the present invention. FIG. 2A shows the structure of the putative carbamate-7. In FIG. 2B, cells treated with carbamate-7 (red) showed a shift in the nuclei/cell distribution over six standard deviations from the control data (blue), in primary screening. FIG. 2C shows that partial dose response curves reveal that carbamate-7 increases the fraction of binucleates at nM concentrations. In FIG. 2D, results from synthetic lethality experiments show a statistically significant difference in the average number of nuclei/cell between untreated and treated samples in wild-type and kif12 null strains (**p<0.0001), but not myoII or racE null strains. Error bars represent SEM.

FIGS. 3(A-D) are a set of images and graphs showing that myosin II cortical dynamics affected by treatment with carbamate-7 according to one embodiment of the present invention. FIG. 3A: Structural Illuminated Micrographs of myoII:GFP myoII cells show an increase in the amount and variability of myosin II bipolar thick filaments in 500-nM carbamate-7 treated (right panels) versus untreated (left panels) cells. In both, the white box represents a zoomed in region, shown to the right of the main images. FIG. 3B: Total Internal Reflection Microscopy (TIRF) images of cells treated with increasing amounts of carbamate-7 show increase of cortical GFP-myosin II, quantified in FIG. 3C. FIG. 3D: Sedimentation assay shows increase of non-monomeric myosin II in 700-nM carbamate-7 treated over untreated cells (n=3). FIG. 3E: Cortical tension measurements show a 1.4-fold increase in cells acutely treated with carbamate-7. Error bars represent SEM.

FIGS. 4(A-G) are a set of images and graphs showing that 4-hydroxyacetophenone activates myosin II. FIG. 4A: Carbamate-7 degrades in DMSO to give three distinct chemical species—3,4-dichloroaniline (3,4-DCA), 4-hydroxacetophenone (4-HAP), and 1,2-bis-(3,4-dichloro-phenyl)-urea. FIG. 4B: Both 3,4-DCA and 4-HAP are required for the shift in binucleation observed from mixtures of carbamate-7 in DMSO, obtained commercially from ChemBridge (CB) and synthesized (syn) in house. FIG. 4C: Myosin II is enriched at the cortex in 4-HAP and both samples only. FIG. 4D: Histogram shows the relative myosin II intensities of the cortex to the cytoplasm. FIG. 4E: TIRF images show an increase in the amount and length of GFP-myosin II BTFs. FIG. 4F: 500 nM 4-HAP shows significant localization of GFP-myosin II within 10 minutes of treatment. FIG. 4G: There is a 1.5-fold increase in cortical tension of cells acutely treated with 500 nM 4-HAP. The change in effective tension (T_{eff}) is dependent on myosin II. Neither the myoII or 5456L myosin cells show an increase in T_{eff}. Error bars represent SEM.

FIG. 5 is a set of images and graphs showing that myosin II activation by 4-HAP requires the normal power stroke and ADP-release step. FIG. 5A: TIRF images of GFP-myosin II, GFP-3×Asp, and GFP-3×Ala expressing myoII null cell-lines in DMSO compared to 10 min 500 nM 4-HAP treatment show an increase in BTFs across all three cell-lines. FIG. 5B shows quantification of 4-HAP timecourse. GFP-S1 and GFP-5456L expressing cell-lines showed no changes over untreated samples FIG. 5A over the time-course of the experiment (FIG. 5B, right panel).

FIG. 6 is a diagram showing model of myosin II activation by 4-HAP.

FIG. 7 is a systemic diagram showing PDAC progression likely dependent on changing mechanical landscape.

FIGS. 8(A-E) are a set of images and graphs showing 4-HAP decreases the deformability of human cells and turns the mechanical profile of pancreatic cancer cells to more WT-like mechanics, decreasing their invasive capacity. FIG. 8A: Micrographs from FIG. 8B creep tests show that 4-HAP stiffens the soft HEK293 cells (creep tests at 0.15 nN/μm²); region of aspiration, Lp; radius of pipette, Rp. FIG. 8C: Sedimentation assay shows increases in assembled myosin IIB and IIC in HEK293 cells. FIG. 8D: Similarly, micrographs of aspirated cells show that 4-HAP tunes the deformability of metastatic PDAC, ASPC-1 cells. FIG. 8E: Creep tests demonstrate that the WT pancreatic cell line HPDE is stiffer than the metastatic PDAC cell-line, ASPC-1 and that 4-HAP stiffens ASPC-1 cells, shifting them towards HPDE-like mechanics (creep tests at 0.25 nN/μm²); region of aspiration, Lp; radius of pipette, Rp. FIG. 8F: 4-HAP increases assembled myosin IIC in ASPC-1 cells, and HPDE cells (FIG. 15H); n provided on bars; *p=0.04, **p=0.007, ***p=0.005. FIG. 8G: 4-HAP does not alter the cortical tension of HL-60 cells which lack the myosin IIB and IIC paralogs. All experiments presented here were performed using cell treated with 500 nM 4-HAP for 1 hr. Migration (FIG. 8H) and invasion (FIG. 8I) assays of ASPC-1 cells show a dose-dependent decrease upon 4-HAP treatment. n provided on bars; **p<0.0001, *p=0.01 for migration assay; *p=0.02 for invasion assay.

FIGS. 9(A-I) are diagrams and graphs showing that CIMPAQ processes high-throughput data and identifies cytokinesis inhibitors. FIG. 9A: Overview workflow diagram of primary screening from 384-well plating to raw data acquisition to CIMPAQ image conversion by segmentation. CIMPAQ analyzes the segmented data to identify and rank-order cytokinesis inhibitors, mitotic inhibitors, and lethal compounds. FIGS. 9(B-D): Plate type affects screening quality. Primary pilot screening was performed on COP plates (FIG. 9D), which showed a tighter distribution of multinucleate cells to mononucleate cells, as well as a tighter distribution of binucleate cells to mononucleate cells as compared to 96-well (FIG. 9B) and 384-well (FIG. 9C) Corning plates. The tighter distribution of untreated WT wells allowed for cytokinesis hits to be more readily identified in the following process: acquisition of (FIG. 9E) raw images of NLS-tdTomato expressing cells and conversion into (FIG. 9F) CIMPAQ-processed version. In both, the white box represents a zoomed quadrant, highlighting both the nuclear and cellular boundaries of a multinucleate (4 nuclei/cell) and several mononucleate cells. FIG. 9G: Sample histogram of a single well showing the distribution of nuclei per cell counts demonstrating high agreement between manual counts and CIMPAQ analysis. Over 50,000 cells have been manually counted to cross compare with CIMPAQ output. FIG. 9H: The Cartesian coordinates defined by the ratio of binucleate (2 nuclei/cell) to mononucleate cells and the ratio of multinucleate (>2 nuclei/cell) to mononucleate cells of the untreated WT wells are fitted to a two dimensional Gaussian distribution. From this distribution, contour lines for all standard deviations from the control mean are determined for a given plate (FIG. 9I). Each blue dot represents one untreated control well from a 384-well plate.

FIGS. 10(A-F) are diagrams and graphs showing validation of CIMPAQ efficiency for cytokinesis and mitotic inhibitors. FIG. 10A: CIMPAQ identified 86% of wells plated with cortexillin I null cells, which are deficient in cytokinesis (cortI null wells, red; WT wells, blue). FIG. 10B: A sample CIMPAQ plot of hit compound (red) from the primary screen of the BIOMOL kinase collection, which is ranked 4 standard deviations away from the control data (blue). FIGS. 10(D-F): CIMPAQ uses a threshold value for nuclear area to identify mitotic inhibitors. FIG. 10D: Raw images of 10 μM nocodazole-treated cells are processed by CIMPAQ (FIG. 10E). FIG. 10F: CIMPAQ uses a simple threshold of 28 pixels for the mean nuclear area to identify early mitotic inhibitors. Distributions of the nuclear area of untreated cells (dark gray), 5 μM nocodazole-treated cells (medium gray, middle), and 10 μM nocodazole-treated cells (light gray) are shown.

FIGS. 11(A-D) are figures and graphs showing characterization of carbamate-7 degradation. FIG. 11A: Degradation of carbamate-7 produces 3,4-dichloroaniline (3,4-DCA), 4-hydroxyacetophenone (4-HAP) and N,N′-bis(3,4-dichlorophenyl)urea (urea). FIG. 11B: HPLC stack plot showing degradation of synthetic and commercial (Source—Chembridge) carbamate-7 in DMSO, and comparison of degradation products to authentic 3,4-DCA and 4-HAP. FIG. 11C: Comparison of the urea degradation product to authentic N,N′-bis(3,4-dichlorophenyl)urea by HPLC analysis. The presence of the urea was also confirmed by mass spectrometry analysis (FIG. 11C, inset) which shows the characteristic isotopic distribution for N,N′-bis(3,4-dichlorophenyl)urea. FIG. 11D: Full nuclei per cell distribution of carbamate-7 and breakdown products. 3,4-DCA and 4-HAP together show an increase in binucleates and a decrease in mononucleates, consistent with the results from C-7 treatment (CB: ChemBridge; syn: synthesized). Compound concentrations (nM): 1, 500, 1000, 5000. 5000 nM 4-HAP was lethal and is therefore not shown. n=400-1441 cells/condition.

FIGS. 12(A-B) are a set of graphs showing reversibility of 4-HAP effect on myosin II cortical enrichment. FIG. 12A: Cells treated with 500 nM 4-HAP had a 2-fold increase in myosin II localization at the cortex by TIRF imaging within 10 min. 500 nM 4-HAP was added at t=−10 min. When the 4-HAP-containing media was removed (t=0), myosin II localization reverts to pre-treatment levels within 15 min of removal. n=20-26 cells per time point. FIG. 12B: Dot plot of the raw data shows the fold-change over the DMSO control at each time point of the washout experiment (left panel), and a dot plot of the raw data of the cell surface contact area for the washout experiments shows no change in surface area among the time points (right panel).

FIGS. 13(A-B) are a set of graphs showing quantification of TIRF images which show an increase in myosin II localization in 4-HAP treated cells, independent of area changes. FIG. 13A: Dot plots of the raw data showing the fold-increase over the DMSO control at 7 min of 500 nM 4-HAP treatment, but not in a similar DMSO time course, 500 nM 3,4-DCA time course, or 500 nM 1,3-bis-(3,4-dichloro-phenyl)-urea time course. FIG. 13B: Dot plots of the raw data of the cell-surface contact area shows no change between time points for all compound treatments.

FIGS. 14(A-B) are a set of graphs showing quantification of TIRF images which reveal an increase in myosin II localization upon 4-HAP treatment in GFP3×Ala and GFP3×Asp expressing cells, but not GFPS456L or GFPS1 expressing cells. (A) Dot plots of the raw data show the fold-increase over the DMSO control for GFP3×Ala and GFP3×Asp rescued myoII null cell lines. GFPS456L and GFPS1 show no change in myosin BTF accumulation at the cortex. FIG. 14B: Dot plots of the raw data of the cell-surface contact area shows no change between time points for all compound treatments.

FIGS. 15(A-J) are a set of graphs of in vitro assembly and motility assays and PDAC results that when taken together, suggest that 4-HAP requires an intact myosin II cytoskeletal network and is myosin II-paralog specific. FIG. 15A: Myosin II Dictyostelium ADCT assembly showed no significant change in in vitro assembly with or without purified 14-3-3 in the presence of 3,4-DCA or 4-HAP as compared to the DMSO control (n=6 for DMSO control, n=3 for all others; error bars represent SEM). Mammalian myosin IIA (FIG. 15B) and myosin IIB (FIG. 15C) assembly was unaffected by 3,4-DCA or 4-HAP as compared to DMSO control (n=3; error bars represent SEM). FIG. 15D: In vitro motility assays show no significant effect of 4-HAP or 3,4-DCA on non-muscle myosin IIB velocity. The gliding filament velocity of actin filaments on non-muscle myosin IIB in the presence of 500 nM 4-HAP (n=30), 500 nM 3,4-DCA (n=30), and both compounds in 1:1 ratio (250 nM each, n=60) was measured. A significant change in velocity compared to the DMSO control (n=30, p=0.2-0.4), was not observed. FIGS. 15(E-F): Quantification of TIRF images reveals no myosin II localization change in 4-HAP treated cortI::GFPmyo cells. FIG. 15E: Dot plot of the raw data shows no fold-change over the DMSO control. FIG. 15F: Dot plot of the raw data of the cell-surface contact area shows no change between time points for compound treatments. FIGS. 15(G-H): 4-HAP affects wild type and metastatic pancreatic cells in a myosin II-specific manner. FIG. 15G: 4-HAP decreases the cortical tension of the PDAC A10.7 cells towards a HPDE-like mechanical profile. FIG. 15H: 4-HAP increases assembled myosin IIC in wild type HPDE cells; n provided on bars; *p=0.04. FIG. 15I: 4-HAP shows little effect on myosin IIA phosphorylation (phosphor-Ser1943) in either HPDE or ASPC-1 cells; n provided on bars; p=0.17. FIG. 15J: Viability assay on ASPC-1 cells across five concentrations of 4-HAP (50 nM, 500 nM, 1 μM, 5 μM, 50 μM) shows no difference over DMSO control.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed nonprovisional applications.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. The terms “comprising” and variations thereof do not have a limiting meaning where these terms appear in the description and claims. Accordingly, the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The terminology as set forth herein is for description of the embodiments only and should not be construed as limiting of the invention as a whole.

As used herein, the term “subject” or “individual” refers to a human or other vertebrate animal. It is intended that the term encompass “patients.”

The term “pharmaceutically acceptable” as used herein means that the compound or composition or carrier is suitable for administration to a subject to achieve the treatments described herein, without unduly deleterious side effects in light of the necessity of the treatment.

The term “therapeutically effective amount” or “pharmaceutically appropriate dosage”, as used herein, refers to the amount of the compounds or dosages that will elicit the biological or medical response of a subject, tissue or cell that is being sought by the researcher, veterinarian, medical doctor or other clinician.

As used herein, “pharmaceutically-acceptable carrier” includes any and all dry powder, solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds of the present invention. Examples of such carriers include, various lactose, mannitol, oils such as corn oil, buffers such as PBS, saline, polyethylene glycol, glycerin, polypropylene glycol, dimethylsulfoxide, an amide such as dimethylacetamide, a protein such as albumin, and a detergent such as Tween 80, mono- and oligopolysaccharides such as glucose, lactose, cyclodextrins and starch.

The term “administering” or “administration”, as used herein, refers to providing the compound or pharmaceutical composition of the invention to a subject suffering from or at risk of the diseases or conditions to be treated or prevented.

The term “systemic delivery”, as used herein, refers to any suitable administration methods which may delivery the compounds in the present invention systemically. In one embodiment, systemic delivery may be selected from the group consisting of oral, parenteral, intranasal, inhaler, sublingual, rectal, and transdermal administrations.

A route of administration in pharmacology and toxicology is the path by which a drug, fluid, poison, or other substance is taken into the body. Routes of administration may be generally classified by the location at which the substance is applied. Common examples may include oral and intravenous administration. Routes can also be classified based on where the target of action is. Action may be topical (local), enteral (system-wide effect, but delivered through the gastrointestinal tract), or parenteral (systemic action, but delivered by routes other than the GI tract), via lung by inhalation.

A topical administration emphasizes local effect, and substance is applied directly where its action is desired. Sometimes, however, the term topical may be defined as applied to a localized area of the body or to the surface of a body part, without necessarily involving target effect of the substance, making the classification rather a variant of the classification based on application location. In an enteral administration, the desired effect is systemic (non-local), substance is given via the digestive tract. In a parenteral administration, the desired effect is systemic, and substance is given by routes other than the digestive tract.

The examples for topical administrations may include epicutaneous (application onto the skin), e.g., allergy testing or typical local anesthesia, inhalational, e.g. asthma medications, enema, e.g., contrast media for imaging of the bowel, eye drops (onto the conjunctiva), e.g., antibiotics for conjunctivitis, ear drops, such as antibiotics and corticosteroids for otitis externa, and those through mucous membranes in the body.

Enteral administration may be administration that involves any part of the gastrointestinal tract and has systemic effects. The examples may include those by mouth (orally), many drugs as tablets, capsules, or drops, those by gastric feeding tube, duodenal feeding tube, or gastrostomy, many drugs and enteral nutrition, and those rectally, various drugs in suppository.

The examples for parenteral administrations may include intravenous (into a vein), e.g. many drugs, total parenteral nutrition intra-arterial (into an artery), e.g., vasodilator drugs in the treatment of vasospasm and thrombolytic drugs for treatment of embolism, intraosseous infusion (into the bone marrow), intra-muscular, intracerebral (into the brain parenchyma), intracerebroventricular (into cerebral ventricular system), intrathecal (an injection into the spinal canal), and subcutaneous (under the skin). Among them, intraosseous infusion is, in effect, an indirect intravenous access because the bone marrow drains directly into the venous system. Intraosseous infusion may be occasionally used for drugs and fluids in emergency medicine and pediatrics when intravenous access is difficult.

Any route of administration may be suitable for the present invention. In one embodiment, the compound of the present invention may be administered to the subject via intravenous injection. In another embodiment, the compounds of the present invention may be administered to the subject via any other suitable systemic deliveries, such as oral, parenteral, intranasal, sublingual, rectal, or transdermal administrations.

In another embodiment, the compounds of the present invention may be administered to the subject via nasal systems or mouth through, e.g., inhalation.

In another embodiment, the compounds of the present invention may be administered to the subject via intraperitoneal injection or IP injection.

As used herein, the term “intraperitoneal injection” or “IP injection” refers to the injection of a substance into the peritoneum (body cavity). IP injection is more often applied to animals than to humans. In general, IP injection may be preferred when large amounts of blood replacement fluids are needed, or when low blood pressure or other problems prevent the use of a suitable blood vessel for intravenous injection.

In animals, IP injection is used predominantly in veterinary medicine and animal testing for the administration of systemic drugs and fluids due to the ease of administration compared with other parenteral methods.

In humans, the method of IP injection is widely used to administer chemotherapy drugs to treat some cancers, in particular ovarian cancer. Although controversial, this specific use has been recommended as a standard of care.

As used herein, the term “Dictyostelium discoideum” refers to a species of soil-living amoeba belonging to the phylum Mycetozoa. Commonly referred to as cellular slime mold, D. discoideum is a eukaryote that transitions from a collection of unicellular amoebae into a multicellular slug and then into a fruiting body within its lifetime. D. discoideum has a unique asexual lifecycle that consists of four stages: vegetative, aggregation, migration, and culmination. The life cycle of D. discoideum is relatively short, which allows for timely viewing of all life stages. The cells involved in the life cycle undergo movement, chemical signaling, and development, which are applicable to human cancer research. The simplicity of its life cycle makes D. discoideum a valuable model organism to study genetic, cellular, and biochemical processes in other organisms. In the present invention, Applicants use Dictyostelium discoideum as a model for cytokinesis. This simple protozoan performs cytokinesis and cell motility in a manner similar to human cells yet it is tractable for genetic, molecular, biochemical, and biophysical methods.

As used herein, the term “cytokinesis” refers to the process in which the cytoplasm of a single eukaryotic cell is divided to form two daughter cells. It usually initiates during the early stages of mitosis, and sometimes meiosis, splitting a mitotic cell in two, to ensure that chromosome number is maintained from one generation to the next. After cytokinesis two (daughter) cells will be formed that enter interphase to make exact copies of the (parent) original cell. In one aspect of the invention, Applicants use cytokinesis as a highly mechanical cell-shape change process to establish an in vivo, large-scale, high-throughput chemical screen for small molecule modulators of cell shape change.

As used herein, the term “myosin II”, also known as conventional myosin, refers to the myosin type responsible for producing contraction, including in nonmuscle and muscle cells. Myosin II contains two heavy chains, each about 2000 amino acids in length, which constitute the head and tail domains. Each of these heavy chains contains the N-terminal head domain, while the C-terminal tails have a coiled-coil structure, which hold the two heavy chains together. Thus, myosin II has two heads. The intermediate neck domain is the region creating the angle between the head and tail. In nonmuscle cells, myosin II has three paralogs: myosin IIA (MYH9), myosin IIB (MYH10), and myosin IIC (MYH14). In smooth muscle, a single gene (MYH11) codes for the heavy chain of myosin II, but splice variants of this gene result in four distinct isoforms. Other myosin II paralogous proteins are found in cardiac and skeletal muscle.

Myosin II may also contain 4 light chains, resulting in 2 per head, weighing 20 (MLC₂₀) and 17 (MLC₁₇) kDa. These bind the heavy chains in the “neck” region between the head and tail. The MLC₂₀ is also known as the regulatory light chain and actively participates in muscle contraction. The MLC₁₇ is also known as the essential light chain. Its exact function is unclear, but is believed to contribute to the structural stability of the myosin, head along with MLC₂₀. Two variants of MLC₁₇ (MLC_{17a/b}) exist as a result of alternate splicing at the MLC₁₇ gene. In muscle cells, the long coiled-coil tails of the individual myosin molecules join together, forming the thick filaments of the sarcomere. The force-producing head domains stick out from the side of the thick filament, ready to walk along the adjacent actin-based thin filaments in response to the proper chemical signals.

As used herein, the term “cell mechanics” refers to a study of the structure and function of biological systems such as cells by means of the methods of mechanics.

As used herein, the term “mechanotransduction” refers to the process of sensing, transmitting, and converting physical forces into biochemical signals and integrating these signals into the cellular responses. Mechanotransduction generally refers to the many mechanisms by which cells convert mechanical stimulus into chemical activity. Mechanotransduction is responsible for a number of senses and physiological processes in the body, including proprioception, touch, balance, and hearing. At the cellular level, mechanotransduction is responsible for guiding processes such as cellular decision making, cell differentiation, and cell morphogenesis. The basic mechanism of mechanotransduction involves converting mechanical signals into electrical or chemical signals. For mechanochemical conversion, mechanical forces are transmitted through the plasma membrane through membrane-actin anchoring proteins and then propagated onto the cytoskeletal networks. Myosin II proteins along with other actin associated proteins are essential components of the mechanotransduction system. These proteins then can lead to the accumulation and/or activation of signaling molecules, including regulators of small GTPases and kinases, allowing for the mechanochemical conversion. For electrical signals, a mechanically gated ion channel makes it possible for sound, pressure, or movement to cause a change in the excitability of specialized sensory cells and sensory neurons. The stimulation of a mechanoreceptor causes mechanically sensitive ion channels to open and produce a transduction current that changes the membrane potential of the cell. Cellular responses to mechanotransduction are variable and give rise to a variety of changes and sensations that extend from molecular to cellular to tissue to organ and organ system levels.

As used herein, the term “derivative” refers to a substance which comprises the same basic carbon skeleton and functionality as the parent compound, but can also bear one or more substituents or substitutions of the parent compound. The derivative may also include salts, solvates and pro-drugs of compounds of the invention.

As used herein, the term “constituent” refers to a substance or a mixture of substances, which are produced during a biochemical or chemical reaction (e.g., decomposition) of another precursor compound. In one specific embodiment of the present invention, the precursor compound is compound (I) or its derivatives.

### II. The Invention

In one embodiment, the present invention discloses small molecules which may be used as activators of myosin II. These small molecules may promote myosin II activity and accumulation through modulation of motor mechanochemistry, assembly and sub-cellular localization pathways. These small molecules may be used to modulate cell and tissue mechanics. This class of molecules, which activate the contractile system of the cell, may be used for therapeutic and tissue engineering applications.

In one embodiment of the present invention, one of the myosin II activators is 4-acetylphenyl-(3,4-dichlorophenyl) carbamate, also named carbamate-7 (C7) (Formula I).

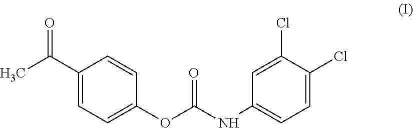 [SMILES: CC(=O)c1ccc(OC(=O)Nc2ccc(Cl)c(Cl)c2)cc1]

Example 2 shows that carbamate-7 may be used as a cytokinesis inhibitor affecting the myosin II-RacE pathway. The experimental results show that carbamate-7 may increase the fraction of binucleates at nM concentrations. Therefore, carbamate-7, or its derivatives or a mixture of their constituents may be used as a myosin II activator. Applicants' initial experiments on carbamate-7 suggested that it targets a key cytokinesis regulatory pathway.

In one embodiment, the present invention discloses a method for modulating cell mechanics of a disease condition in a subject comprising the step of administering an effective amount of a compound having the formula (I).

Any suitable administering method may be used in the present invention. In one embodiment, carbamate-7, or its derivatives or a mixture of their constituents may be administered by systemic delivery. In one specific embodiment, the method of administering by systemic delivery is selected from the group consisting of oral, parenteral, intranasal, sublingual, rectal, and transdermal administration.

In another embodiment, the present invention discloses a compound having formula I for use in activating myosin II to treat a disease condition in a subject by systemic delivery. Applicants envision that carbamate-7, or its derivatives or a mixture of their constituents, may be used in a combination of other known myosin II modulating compounds to modulate myosin II and activate it in the cell. Some of the exemplary myosin II modulating compounds may include Omecamtiv mecarbil (Cytokinetics, INC.), Blebbistatin, B D M, Calyculin A, Myosin light chain phosphorylation inhibitors including myosin light chain kinase (MLCK) inhibitors, such as ML-7, and Rho kinase (ROCK) inhibitors, such as Y-27632.

In one embodiment, the myosin II activator is 4-hydroxyacetophenone (4-HAP) (Formula II), or its derivatives or a mixture of their constituents.

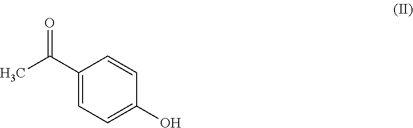

In one embodiment, the myosin II activator may include any compounds which can produce 4-HAP (Formula II) or its derivatives as one of the constituents upon decomposition of the compound.

Applicants' initial experiments (Example 4) show that carbamate-7 is unstable, which can degrade rapidly to form two major products, 4-hydroxyacetophenone (4-HAP) (Formula II) and 3,4-dichloroaniline (3,4-DCA) (Formula III).

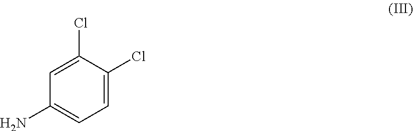

As shown in Examples 4 and 5, 4-HAP or its derivatives can increase the cortical localization of the mechanoenzyme myosin II, thereby increasing the cell's cortical tension. Activity of 4-HAP is independent of myosin heavy-chain phosphorylation, the primary regulator of bipolar thick-filament assembly. Furthermore, similar effects on myosin recruitment have been observed in mammalian cells, suggesting that 4-HAP or its derivatives may pharmacologically modify cell mechanics across phylogeny and disease states.

In one embodiment, the present invention discloses a method for modulating cell mechanics of a disease condition in a subject comprising the step of administering an effective amount of a compound having the formula (II).

Any suitable administering method may be used in the present invention. In one embodiment, 4-HAP or its derivatives may be administered by systemic delivery. In one specific embodiment, the method of administering by systemic delivery is selected from the group consisting of oral, parenteral, intranasal, sublingual, rectal, and transdermal administration.

In another embodiment, the present invention discloses a compound of 4-HAP or its derivatives having Formula II for use in activating myosin II to treat a disease condition in a subject by systemic delivery.

In one embodiment, the active compound of 4-HAP or its derivatives may be combined with other compounds for activating myosin II to treat a disease condition in a subject by systemic delivery.

For example, 3,4-dichloroaniline (3,4-DCA) by itself appears to have limited cellular effect. But, to have maximal cytokinesis inhibition, 3,4-DCA and 4-HAP or its derivatives work additively. Thus, 4-HAP or its derivatives may be used by itself or in combination with 3,4-DCA to differentially modulate cell division.

In one embodiment, Applicants envision that 4-HAP or its derivatives may be used in a combination with any other myosin II modulating compounds to modulate myosin II and activate it in the cell. 4-HAP or its derivatives may also be used with any other known myosin II modulating compounds. Some of the exemplary myosin II modulating compound may include Omecamtiv mecarbil (Cytokinetics, INC.), Blebbistatin, BDM, Calyculin A, Myosin light chain phosphorylation inhibitors including myosin light chain kinase (MLCK) inhibitors, such as ML-7, and Rho kinase (ROCK) inhibitors, such as Y-27632. Applicants envision that 4-HAP may be used in combination with other compounds that target other aspects of cell signaling, membrane receptors, ion channels, any of which target other cell and tissue related behaviors, including, but not limited to, cell growth, motility, migration, and invasion.

In one embodiment, the present invention discloses a method for modulating cell mechanics of a disease condition in a subject comprising administering by systemic delivery effective amounts of compounds 4-HAP or its derivatives and 3,4-DCA having the formula (II) and formula (III), respectively. In one embodiment, both compounds 4-HAP and 3,4-DCA may be administered at the same time. Effective amounts of compounds 4-HAP and 3,4-DCA may be initially mixed. The mixture may subsequently be administered by any suitable systemic delivery methods. In another embodiment, effective amounts of compounds 4-HAP or its derivatives and 3,4-DCA may be individually administered by any suitable systemic delivery methods.

In one embodiment, the present invention also discloses other small molecule compounds which may be used as myosin II activators and/or and cytokinesis modulators. Using the Dictyostelium Drug Discovery Platform (3DP), Applicants have identified other small molecule compounds as cytokinesis modulators. For example, 4-phenyl-2-butanone (4-nitrophenyl) hydrazone (Formula IV), may also inhibit cell division but through a different pathway from those of 4-HAP. A genetic selection for suppressors of 4-phenyl-2-butanone (4-nitrophenyl) hydrazone inhibition identified ATP synthase β-subunit as a genetic suppressor, which is particularly interesting as angiostatins are known to target F₁F₀ ATP synthase.

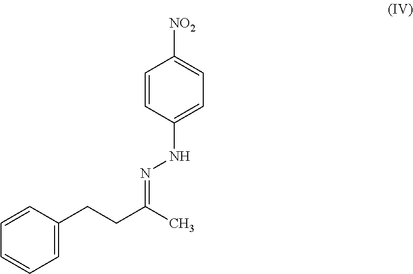 [SMILES: C/C(CCc1ccccc1)=N\Nc1ccc([N+](=O)[O-])cc1]

In one embodiment, the present invention disclose a method for modulating cell mechanics of a disease condition in a subject comprising the step of administering by systemic delivery an effective amount of a compound having the formula (IV).

Any suitable administering method may be used in the present invention. In one embodiment, 4-phenyl-2-butanone (4-nitrophenyl) hydrazone may be administered by systemic delivery. In one specific embodiment, the method of administering by systemic delivery is selected from the group consisting of oral, parenteral, intranasal, sublingual, rectal, and transdermal administration.

In one embodiment, Applicants envision that 4-phenyl-2-butanone (4-nitrophenyl) hydrazone may be used in a combination with any other myosin II modulating compounds to modulate myosin II and activate it in the cell. For example, 4-phenyl-2-butanone (4-nitrophenyl) hydrazone may be combined with carbamate-7, or its derivatives or a mixture of their constituents, or 4-HAP or its derivatives as discussed above to modulate myosin II and activate it in the cell. 4-phenyl-2-butanone (4-nitrophenyl) hydrazone may also be used with any other known myosin II modulating compounds. Some of the exemplary myosin II modulating compound may include Omecamtiv mecarbil (Cytokinetics, INC.), Blebbistatin, BDM, Calyculin A, Myosin light chain phosphorylation inhibitors including myosin light chain kinase (MLCK) inhibitors, such as ML-7, and Rho kinase (ROCK) inhibitors, such as Y-27632.

The present invention also encloses pharmaceutical compositions comprising one or more active compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds of the present invention may be incorporated into transdermal patches designed to deliver the appropriate amount of the drug in a continuous fashion.

For preparing solid compositions such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a compound of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. The tablets or pills of the novel composition can be coated or otherwise compounded to provide a dosage affording the advantage of prolonged action. For example, the tablet or pill can comprise an inner dosage and an outer dosage component, the latter being in the form of an envelope over the former. The two components can be separated by an enteric layer which, serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The compounds of the present invention are particularly useful when formulated in the form of a pharmaceutical injectable dosage, including a compound described and claimed herein in combination with an injectable carrier system. As used herein, injectable and infusion dosage forms (i.e., parenteral dosage forms) include, but are not limited to, liposomal injectables or a lipid bilayer vesicle having phospholipids that encapsulate an active drug substance. Injection includes a sterile preparation intended for parenteral use.

Five distinct classes of injections exist as defined by the USP: emulsions, lipids, powders, solutions and suspensions. Emulsion injection includes an emulsion comprising a sterile, pyrogen-free preparation intended to be administered parenterally. Lipid complex and powder for solution injection are sterile preparations intended for reconstitution to form a solution for parenteral use. Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization. Solution injection involves a liquid preparation containing one or more drug substances dissolved in a suitable solvent or mixture of mutually miscible solvents that is suitable for injection.

Solution concentrate injection involves a sterile preparation for parenteral use that, upon addition of suitable solvents, yields a solution conforming in all respects to the requirements for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives.

### Therapeutic Methods

Combinations of the compounds described above may be administered to a subject in a single dosage form or by separate administration of each active agent. The agents may be formulated into a single tablet, pill, capsule, or solution for parenteral administration and the like. Individual therapeutic agents may be isolated from other therapeutic agent(s) in a single dosage form. Formulating the dosage forms in such a way may assist in maintaining the structural integrity of potentially reactive therapeutic agents until they are administered. Therapeutic agents may be contained in segregated regions or distinct caplets or the like housed within a capsule. Therapeutic agents may also be provided in isolated layers in a tablet.

Alternatively, the therapeutic agents may be administered as separate compositions, e.g., as separate tablets or solutions. One or more active agent may be administered at the same time as the other active agent(s) or the active agents may be administered intermittently. The length of time between administrations of the therapeutic agents may be adjusted to achieve the desired therapeutic effect. In certain instances, one or more therapeutic agent(s) may be administered only a few minutes (e.g., about 1, 2, 5, 10, 30, or 60 min) after administration of the other therapeutic agent(s). Alternatively, one or more therapeutic agent(s) may be administered several hours (e.g., about 2, 4, 6, 10, 12, 24, or 36 h) after administration of the other therapeutic agent(s). In certain embodiments, it may be advantageous to administer more than one dosage of one or more therapeutic agent(s) between administrations of the remaining therapeutic agent(s). For example, one therapeutic agent may be administered at 2 hours and then again at 10 hours following administration of the other therapeutic agent(s). The therapeutic effects of each active ingredient should overlap for at least a portion of the duration, so that the overall therapeutic effect of the combination therapy is attributable in part to the combined or synergistic effects of the combination therapy.

The dosage of the active agents will generally be dependent upon a number of factors including pharmacodynamic characteristics of each agent of the combination, mode and route of administration of active agent(s), the health of the patient being treated, the extent of treatment desired, the nature and kind of concurrent therapy, if any, and the frequency of treatment and the nature of the effect desired. In general, dosage ranges of the active agents often range from about 0.001 to about 250 mg/kg body weight per day. However, some variability in this general dosage range may be required depending upon the age and weight of the subject being treated, the intended route of administration, the particular agent being administered and the like. Since two or more different active agents are being used together in a combination therapy, the potency of each agent and the interactive effects achieved using them together must be considered. Importantly, the determination of dosage ranges and optimal dosages for a particular mammal is also well within the ability of one of ordinary skill in the art having the benefit of the instant disclosure.

Dosage ranges for agents may be as low as 5 ng/d. In certain embodiments, about 10 ng/day, about 15 ng/day, about 20 ng/day, about 25 ng/day, about 30 ng/day, about 35 ng/day, about 40 ng/day, about 45 ng/day, about 50 ng/day, about 60 ng/day, about 70 ng/d, about 80 ng/day, about 90 ng/day, about 100 ng/day, about 200 ng/day, about 300 ng/day, about 400 ng/day, about 500 ng/day, about 600 ng/day, about 700 ng/day, about 800 ng/day, about 900 ng/day, about 1 μg/day, about 2 μg/day, about 3 μg/day, about 4 μg/day, about 5 μg/day, about 10 μg/day, about 15 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, about 1 mg/day, about 2 mg/day, about 3 mg/day, about 4 mg/day, about 5 mg/day, about 10 mg/day, about 15 mg/day, about 20 mg/day, about 30 mg/day, about 40 mg/day, or about 50 mg/day of an agent of the invention is administered.

In certain embodiments, the agents of the invention are administered in pM or nM concentrations. In certain embodiments, the agents are administered in about 1 pM, about 2 pM, about 3 pM, about 4 pM, about 5 pM, about 6 pM, about 7 pM, about 8 pM, about 9 pM, about 10 pM, about 20 pM, about 30 pM, about 40 pM, about 50 pM, about 60 pM, about 70 pM, about 80 pM, about 90 pM, about 100 pM, about 200 pM, about 300 pM, about 400 pM, about 500 pM, about 600 pM, about 700 pM, about 800 pM, about 900 pM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, or about 900 nM concentrations. A dosage range of the present compounds for administration to animals, including humans, is from about 0.001 nM to about 500 mM. A preferred dosage range is 0.1 nM to 100 μM. A more preferred dosage range is 1 nM to 10 μM. The most preferred dosage range is 1 nM to 1 μM.

It may be advantageous for the pharmaceutical combination to be comprised of a relatively large amount of the first component compared to the second component. In certain instances, the ratio of the first active agent to second active agent is about 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, 15:1, 10:1, 9:1, 8:1, 7:1, 6:1, or 5:1. It further may be preferable to have a more equal distribution of pharmaceutical agents. In certain instances, the ratio of the first active agent to the second active agent is about 4:1, 3:1, 2:1, 1:1, 1:2, 1:3, or 1:4. It also may be advantageous for the pharmaceutical combination to have a relatively large amount of the second component compared to the first component. In certain instances, the ratio of the second active agent to the first active agent is about 30:1, 20:1, 15:1, 10:1, 9:1, 8:1, 7:1, 6:1, or 5:1. In certain instances, the ratio of the second active agent to first active agent is about 100:1, 90:1, 80:1, 70:1, 60:1, 50:1, or 40:1. In certain instances, the ratio of the second active agent to first active agent is about 200:1, 190:1, 180:1, 170:1, 160:1, 150:1, 140:1, 130:1, 120:1, or 110:1. A composition comprising any of the above-identified combinations of first therapeutic agent and second therapeutic agent may be administered in divided doses about 1, 2, 3, 4, 5, 6, or more times per day or in a form that will provide a rate of release effective to attain the desired results. The dosage form may contain both the first and second active agents. The dosage form may be administered one time per day if it contains both the first and second active agents.

For example, a formulation intended for oral administration to humans may contain from about 0.1 mg to about 5 g of the first therapeutic agent and about 0.1 mg to about 5 g of the second therapeutic agent, both of which are compounded with an appropriate and convenient amount of carrier material varying from about 5 to about 95 percent of the total composition. Unit dosages will generally contain between about 0.5 mg to about 1500 mg of the first therapeutic agent and 0.5 mg to about 1500 mg of the second therapeutic agent. The dosage may be about 25 mg, 50 mg, 100 mg, 200 mg, 300 mg, 400 mg, 500 mg, 600 mg, 800 mg, or 1000 mg, etc., up to about 1500 mg of the first therapeutic agent. The dosage may be about 25 mg, 50 mg, 100 mg, 200 mg, 300 mg, 400 mg, 500 mg, 600 mg, 800 mg, or 1 000 mg, etc., up to about 1500 mg of the second therapeutic agent.

The small molecule compounds, e.g., carbamate-7, 4-hydroxyacetophenone, and 4-phenyl-2-butanone (4-nitrophenyl) hydrazone, are useful to develop drugs that modulate myosin II, activating it in the cell, or modulating cytokinesis, perhaps through the ATP synthase β-subunit. Such compounds will have anti-cancer and/or anti-metastatic potential, be used to guide stem cell differentiation, and/or have therapeutic potential for a host of degenerative diseases such as motor neuron disease.

In one aspect, the present invention discloses an in vivo, large-scale and high-throughput method of screening by targeting cell mechanics to discover novel therapeutics for treating a disease condition related to cell mechanics defects. Applicants appreciate that in a disease condition such as a cancer, altered cell mechanics are a hallmark of metastatic efficiency. Applicants envision that one therapeutic approach is to increase cellular elasticity, which would in turn reduce metastatic potential and act downstream of cancer-inducing genetic alterations. Such chemical modulators will be powerful for a host of other applications of cell and tissue engineering. Additionally, modifications of the described compounds that may be caged and then uncaged in cells may be useful for directing the compounds to particular cells. Such applications might be useful for creating cells within a population that have differential mechanics or alternatively, homogenizing the mechanics of cells within the population.

The screening technology also can be adapted to a host of available mutant cell lines, which can increase the diversity of modulators that may be identified. Further as D. discoideum is an entire organism, this removes the ambiguity of how human cell-lines vary from the normal primary cells and how they become highly divergent between laboratory stocks.

Finally, the screening approach may be used to identify small molecule protectors of cell viability for the protection against toxic chemical agents. For example, one embodiment would be to screen for chemical protectors of smoke, such as from cigarettes, which is the leading cause of chronic obstructive pulmonary disease, the third leading cause of death in the U.S.

Applicants designed a live-cell, high-throughput chemical screen to identify mechanical modulators. Specifically, Applicants use cytokinesis as an evolutionarily conserved, highly mechanical cell-shape change platform to establish an in vivo, large-scale, high-throughput chemical screen for small molecule modulators of cell shape change.

In one embodiment, the present screen method searches for compounds that would provide a correcting function rather than simply killing cells (i.e., do no harm by minimizing side effects). In one embodiment, the present screen method identifies chemicals as highly potent, subtle modulators, rather than those that would completely abolish cell division.

In one embodiment, the present screen method analyzes and identifies compounds on the basis of their cytokinesis inhibitory activity, mitotic inhibitory activity, or lethality. Specifically, the present screen method identifies small molecules as novel cytokinesis inhibitors, mitotic inhibitors, and lethal compounds.

In one embodiment, the screening method comprises the steps of: (a) obtaining cells and place the cells on multiple-well substrate plates for cytokinesis; (b) contacting the cells on multi-well substrate plates with compound candidates; and (c) monitoring and analyzing the cytokinesis of the cells.

Any cell types suitable for analyzing cytokinesis as appreciated by one skilled in the art can be used in the present invention. In one specific embodiment, the cell type may be Dictyostelium discoideum strains. The cells may be placed on a multi-well substrate plate. In one embodiment, a polymer substrate plate with multi-wells may be used. Specifically, multi-well Cyclo Olegin Polymer (COP) plates are used for their optical characteristics that generated a tighter distribution of nuclei/cell counts.

In one preferred embodiment, the cells may be engineered to include nuclear reporters. In one specific embodiment, the nuclear reporters may include NLS-tdTomato which is optimal for live cell imaging in normal growth media over multiple time points, and that allows for the number of nuclei in each cell and nuclear area to be discerned.

The cells on the substrate plate may be contacted with compound candidates. In one specific embodiment, the present screen method is designed to test a large amount of compound candidates. For example, over 22,000 compounds from the ChemBridge Divert-SET library were screened.

The cytokinesis and growth of the cells may then be monitored and analyzed. In one embodiment, the cytokinesis and growth of the cells may then be monitored and analyzed by an imaging technique. A suitable imaging technique may include fluorescence, Raman, UV-Vis, IR or any other imaging technique appreciated by one skilled in the art. In one specific embodiment, the imaging technique is TIRF imaging. In one embodiment, the imaging technique is a confocal imaging technique. In one embodiment, the imaging technique uses a high content imager.

Specifically, Applicants developed a processing and analysis platform called Cytokinesis Image Processing Analysis Quantification (CIMPAQ), to maximize data collection from a single screen and to perform in-house data analysis. In one embodiment, by using CIMPAQ, one can analyze high content imaging data to identify cell viability, and cytokinetic and mitotic defects of Dictyostelium cells. By respectively counting cells, one can further determine the number of nuclei per cell, and measure the nuclear size of the cells. The Examples show the detail of the platform of CIMPAQ and methods of using such a platform. In the original embodiment of CIMPAQ, the program uses a single reporter—NLS-tdTomato—to track the nuclei and cytoplasmic volumes by using watershed to identify the different cell compartments. CIMPAQ can be readily adapted to other reporters that mark structures and organelles at the plasma membrane or cytoplasm in addition to the nucleus for further assay development.

In one embodiment, cytokinesis properties of the cells such as the binucleate to mononucleate ratio, and the multinucleate to mononucleate ratio may be used to determine cytokinesis inhibition of the corresponding compound candidates. For example, an increase in the binucleate to mononucleate ratio, and an increase of the multinucleate to mononucleate ratio may both indicate mild cytokinesis inhibition of the corresponding compounds.

The Examples shows the detail of this method and the live-cell, high-throughput chemical screen. By using the method and the chemical screen, Applicants identify small molecule compounds as mechanical modulators. Specifically, Applicants identify compounds such as 4-hydroxyacetophenone (4-HAP) as discussed above, which enhances the cortical localization of the mechanoenzyme myosin II, independent of myosin heavy-chain phosphorylation, thus increasing cellular cortical tension.

### EXAMPLES

### Example 1

### CIMPAQ Processes of High-Throughput Data and Identification of Mechanical Modulators, Mitotic Inhibitors, and Lethal Compounds

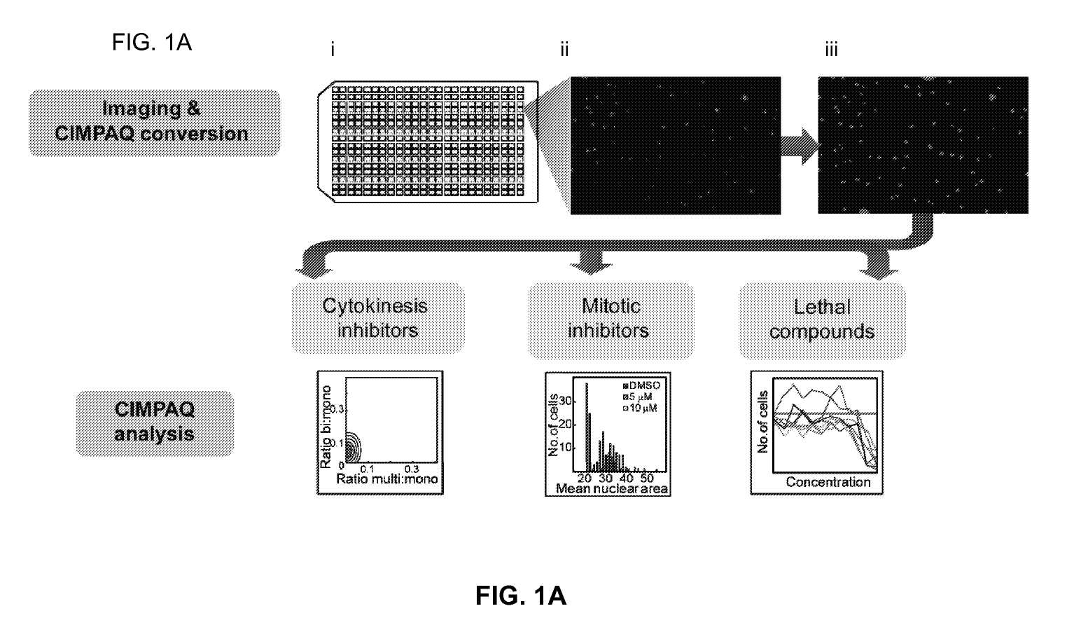

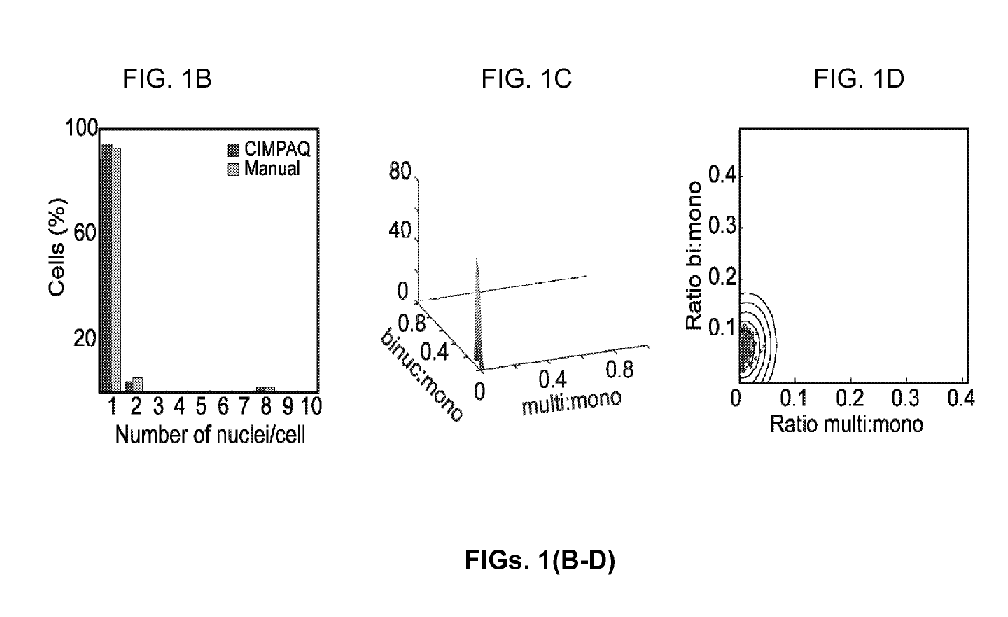

FIGS. 1(A-D) are a set of diagrams and graphs showing CIMPAQ processes of high-throughput data and identification of mechanical modulators, mitotic inhibitors, and lethal compounds. FIG. 1A shows workflow diagram of primary screening from 384-well plating (i) to raw data acquisition (ii) to CIMPAQ image conversion by segmentation (iii). Cytokinesis hits are identified in a 5-step process: Acquisition of FIG. 1A(ii) raw images of NLS-tdTomato expressing cells and conversion into FIG. 1A (iii) CIMPAQ-processed version FIG. 1B shows sample histogram of a single well showing the distribution of nuclei per cell counts demonstrating high agreement between manual counts and CIMPAQ analysis. The Cartesian coordinates defined by the ratio of bi- to mono-nucleated cells and the ratio of multi- to mononucleated cells of the untreated WT wells are fitted to a two dimensional Gaussian distribution in FIG. 1C. From this distribution, contour lines for all standard deviations from the control mean are determined for a given plate as shown in FIG. 1D.

### Example 2

### Identification of Carbamate-7 as a Cytokinesis Inhibitor Affecting the Myosin II-RacE Pathway

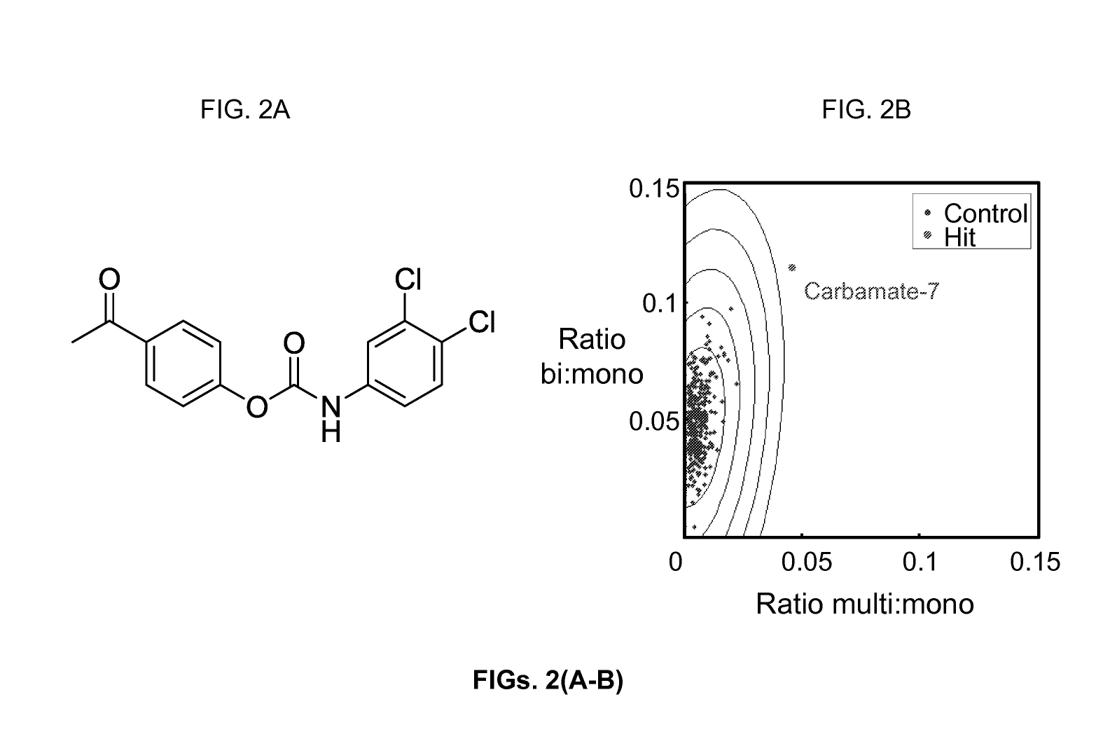

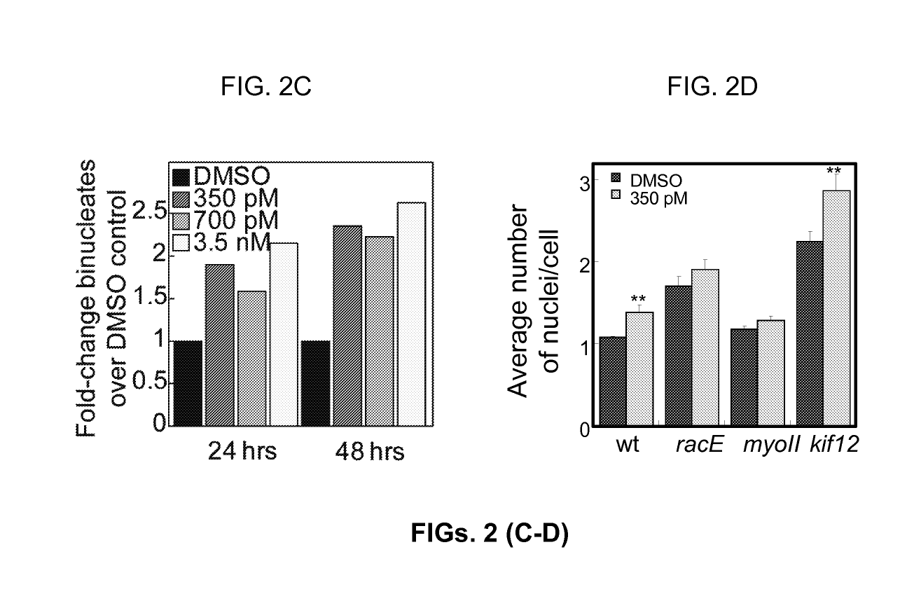

FIGS. 2(A-D) are a set of images and graphs showing the molecular structure of carbamate-7 and identification of carbamate-7 as a cytokinesis inhibitor affecting the myosin II-RacE pathway according to one embodiment of the present invention. FIG. 2A shows the structure of the putative carbamate-7. In FIG. 2B, cells treated with carbamate-7 (red) showed a shift in the nuclei/cell distribution over six standard deviations from the control data (blue), in primary screening. FIG. 2C shows that partial dose response curves reveal that carbamate-7 increases the fraction of binucleates at nM concentrations. In FIG. 2D, results from synthetic lethality experiments show a statistically significant difference in the average number of nuclei/cell between untreated and treated samples in wild-type and kif12 null strains (**p<0.0001), but not myoII or RacE null strains. Error bars represent SEM.

### Example 3

### Myosin II Cortical Dynamics Affected by Treatment with Carbamate-7

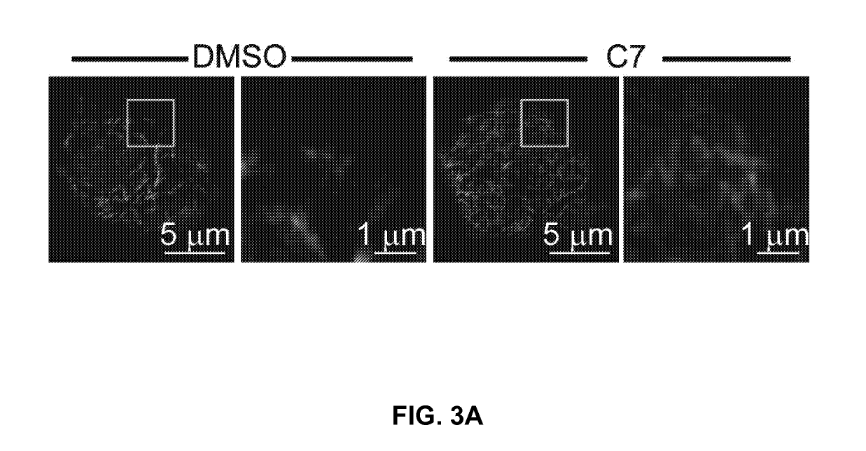

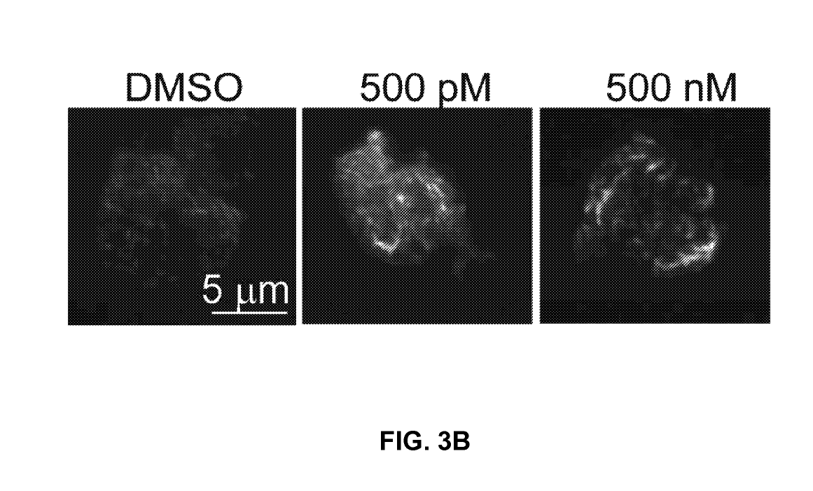

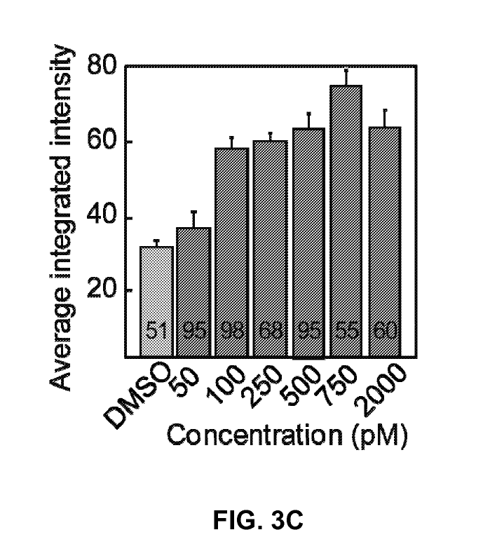

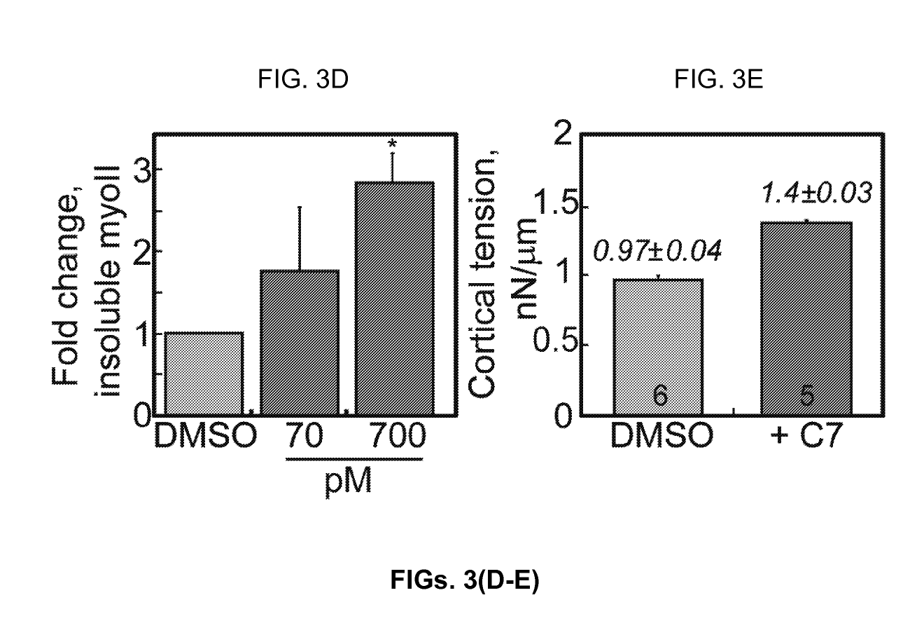

FIGS. 3(A-D) are a set of images and graphs showing that myosin II cortical dynamics affected by treatment with carbamate-7 according to one embodiment of the present invention. FIG. 3A: Structural Illuminated Micrographs of myoII:GFP myoII cells show an increase in the amount and variability of myosin II bipolar thick filaments in 500-nM carbamate-7 treated (right panels) versus untreated (left panels) cells. In both, the white box represents a zoomed in region, shown to the right of the main images. FIG. 3B: Total Internal Reflection Microscopy (TIRF) images of cells treated with increasing amounts of carbamate-7 show increase of cortical GFP-myosin II, quantified in FIG. 3C. FIG. 3D: Sedimentation assay shows increase of non-monomeric myosin II in 700-nM carbamate-7 treated over untreated cells (n=3). FIG. 3E: Cortical tension measurements show a 1.4-fold increase in cells acutely treated with carbamate-7. Error bars represent SEM.

### Example 4

### 4-Hydroxyacetophenone Activates Myosin II

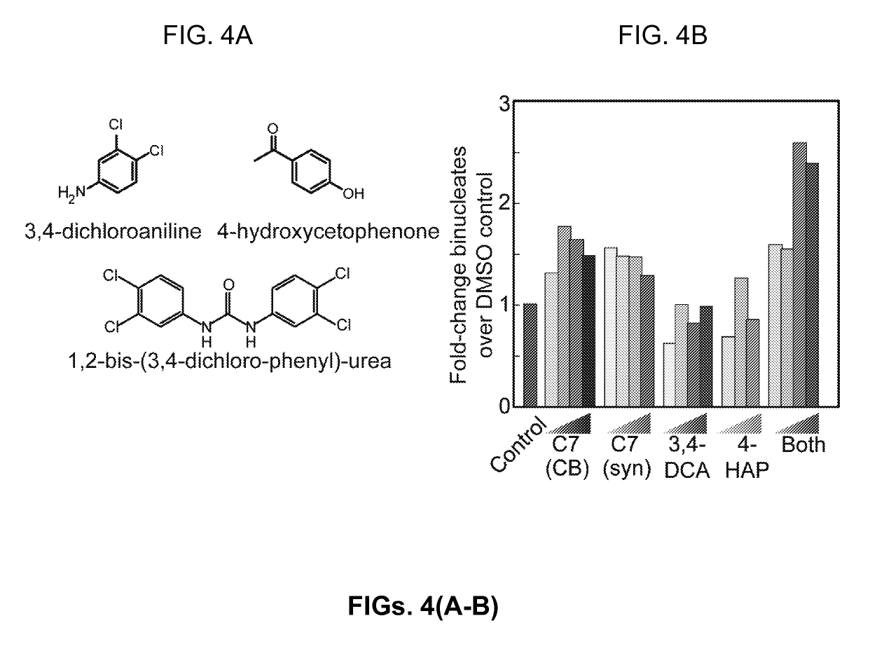

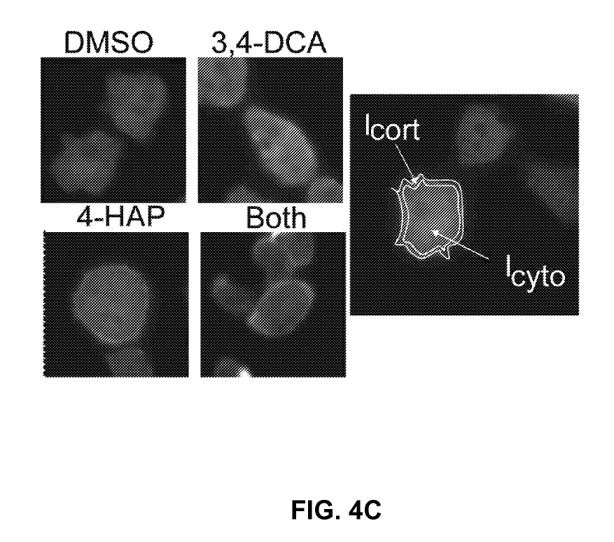

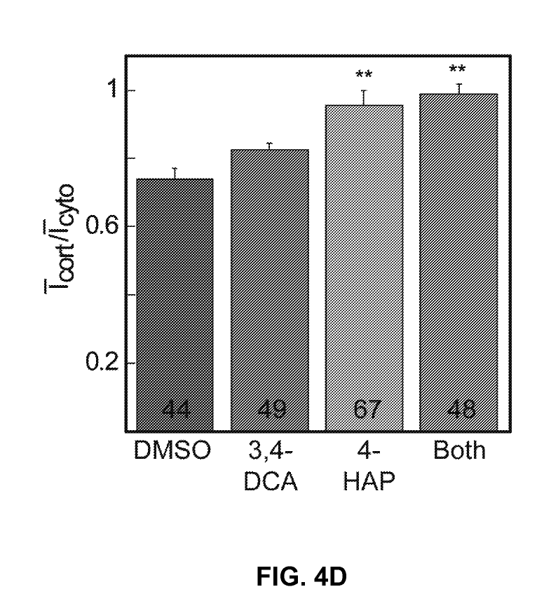

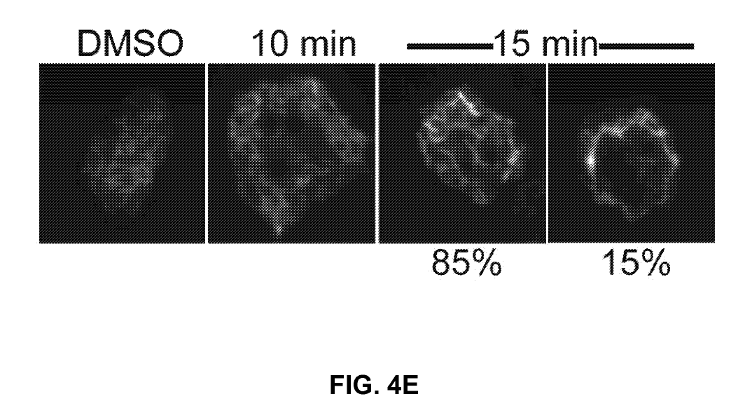

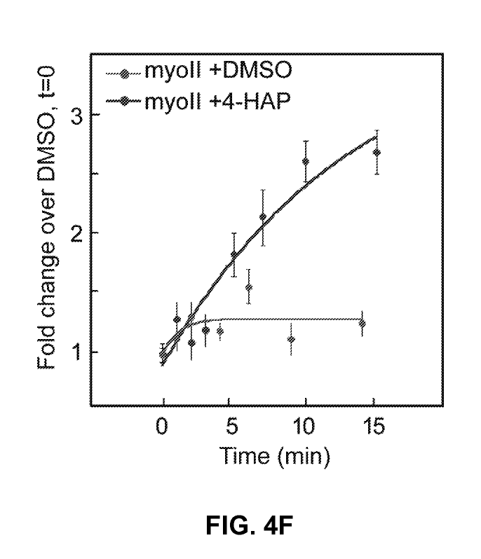

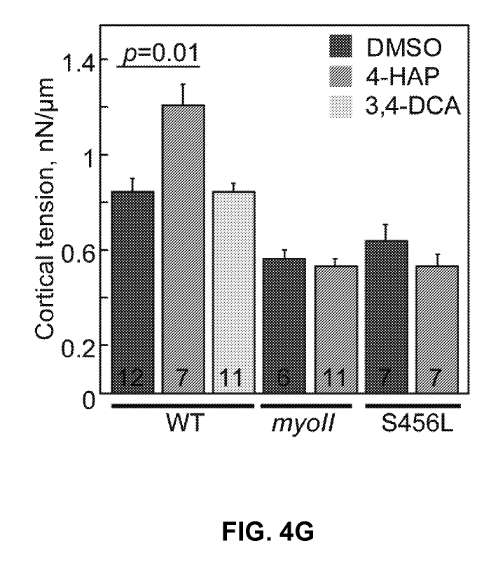

FIGS. 4(A-G) are a set of images and graphs showing that 4-hydroxyacetophenone activates myosin II. FIG. 4A: Carbamate-7 degrades in DMSO to give three distinct chemical species—3,4-dichloroaniline, 4-hydroxacetophenone, and 1,2-bis-(3,4-dichloro-phenyl)-urea. FIG. 4B: Both 3,4-DCA and 4-HAP are required for the shift in binucleation observed from mixtures of carbamate-7 in DMSO, obtained commercially from ChemBridge (CB) and synthesized (syn) in house. FIG. 4C: Myosin II is enriched at the cortex in 4-HAP and both samples only. FIG. 4D: Histogram shows the relative myosin II intensities of the cortex to the cytoplasm. FIG. 4E: TIRF images show an increase in the amount and length of GFP-myosin II BTFs. FIG. 4F: 500 nM 4-HAP shows significant localization of GFP-myosin II within 10 minutes of treatment. FIG. 4G: There is a 1.5-fold increase in cortical tension of cells acutely treated with 500 nM 4-HAP. The change in effective tension (T_{eff}) is dependent on myosin II. Neither the myoII nor S456L myosin cells show an increase in T_{eff}. Error bars represent SEM.

### Example 5

### Myosin II Activation by 4-HAP Requires the Normal Power Stroke and ADP-Release Step

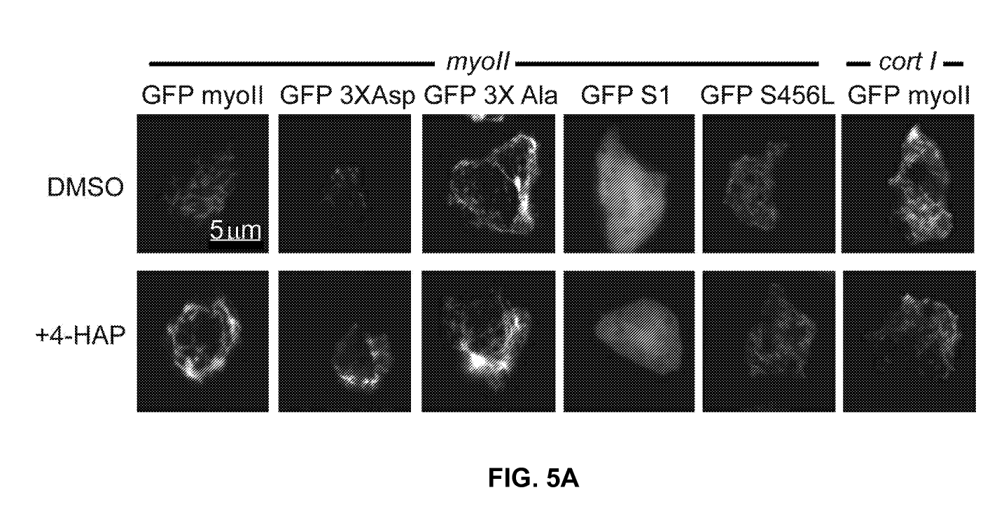

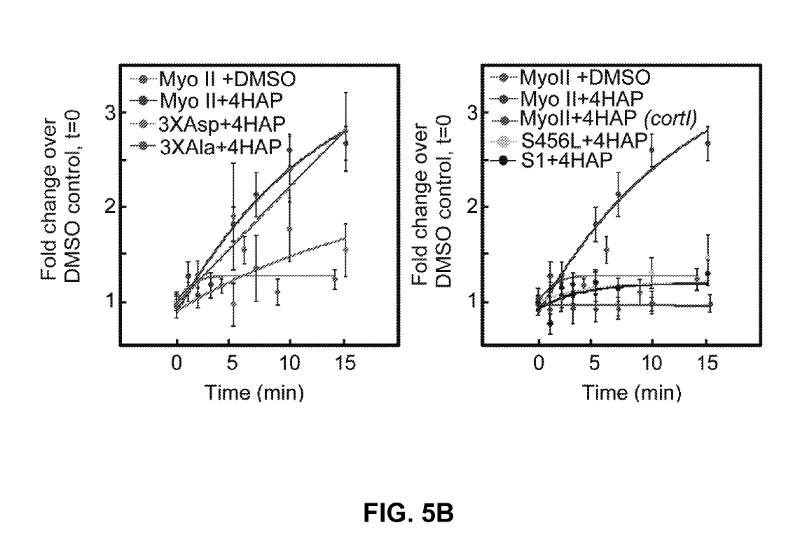

FIG. 5 is a set of images and graphs showing that myosin II activation by 4-HAP requires the normal power stroke and ADP-release step. FIG. 5A: TIRF images of GFP-myosin II, GFP-3×Asp, and GFP-3×Ala expressing myoII null cell-lines in DMSO compared to 10 min 500 nM 4-HAP treatment show an increase in BTFs across all three cell-lines. FIG. 5B shows quantification of 4-HAP time course. GFP-S1 and GFP-S456L expressing cell-lines showed no changes over untreated samples FIG. 5A over the time-course of the experiment (FIG. 5B, right panel).

### Example 6

### Model of Myosin II Activation by 4-HAP

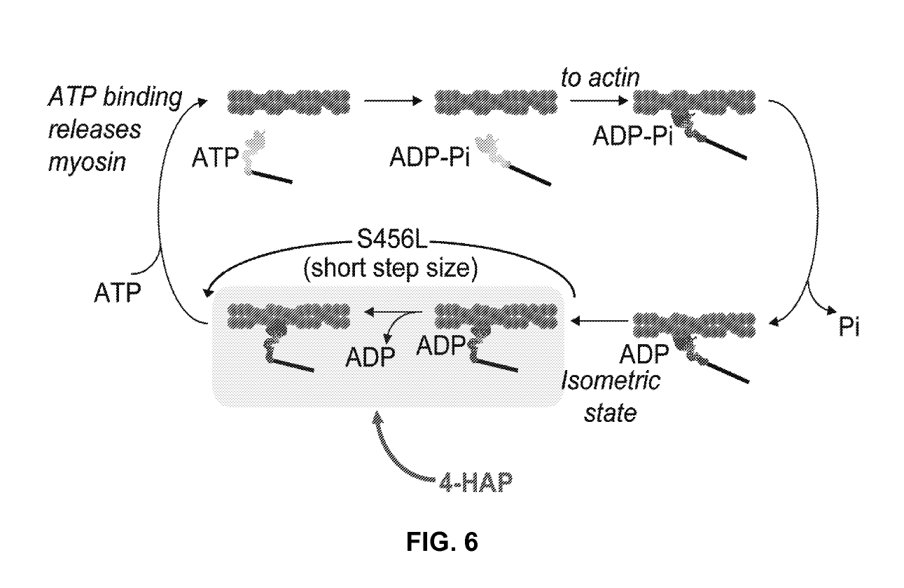

FIG. 6 is a diagram showing model of myosin II activation by 4-HAP.

### Example 7

### PDAC Progression Likely Dependent on Changing Mechanical Landscape

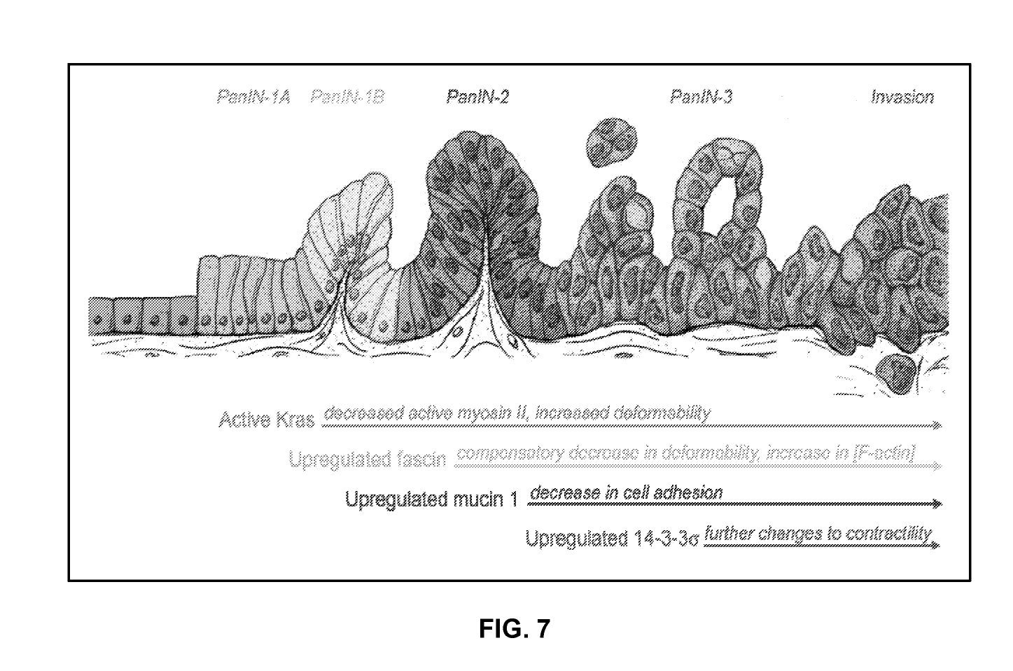

FIG. 7 is a systemic diagram showing PDAC progression likely dependent on changing mechanical landscape.

### Example 8

### 4-HAP Restores PDAC Mechanics Towards Wild Type (WT) Mechanics, Working Through Myosin IIB and IIC

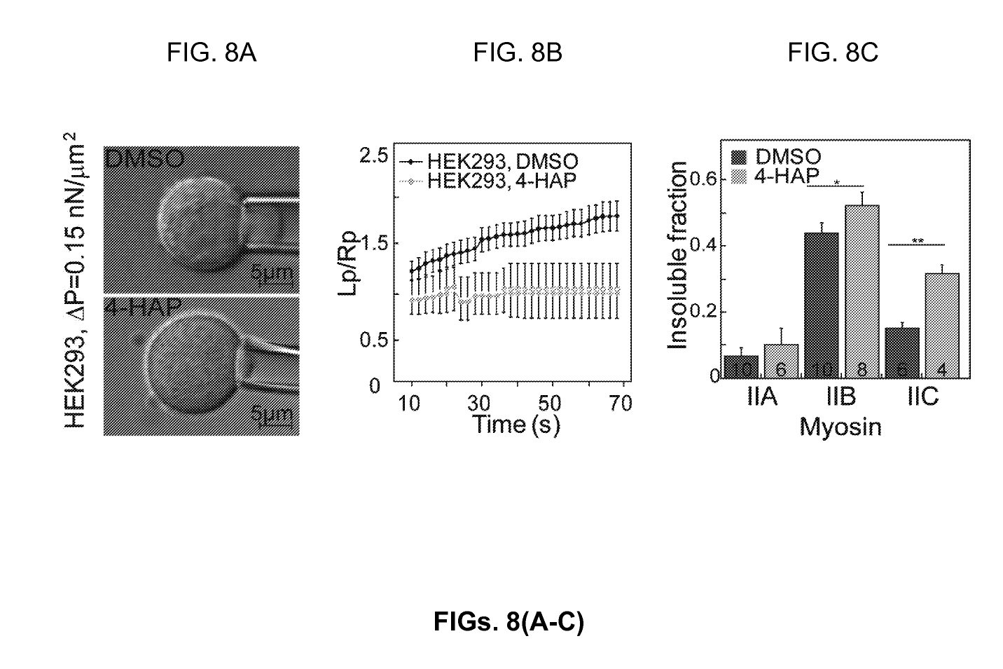

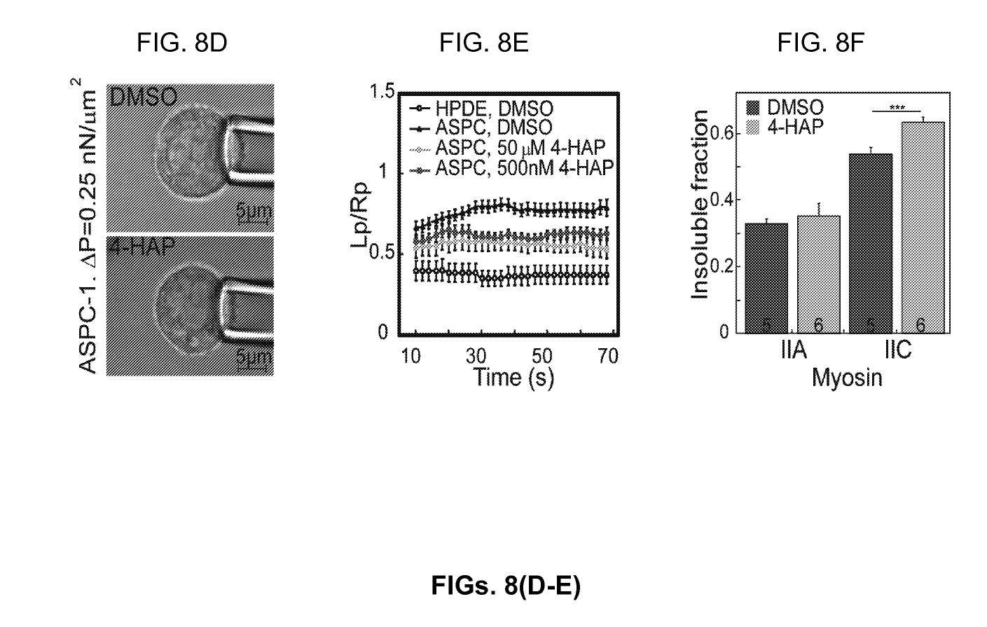

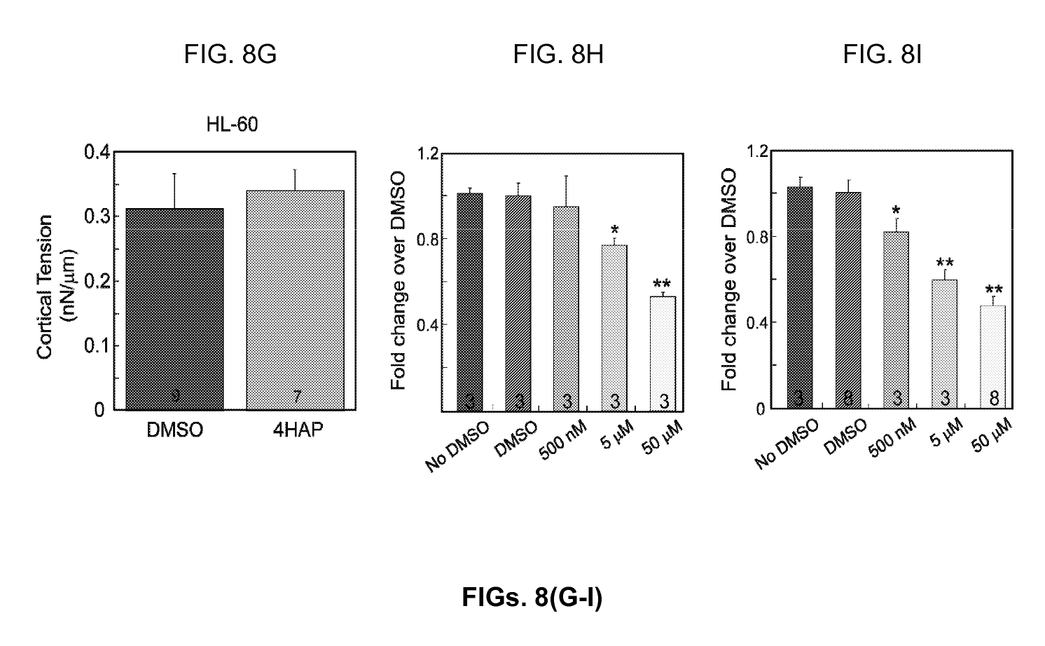

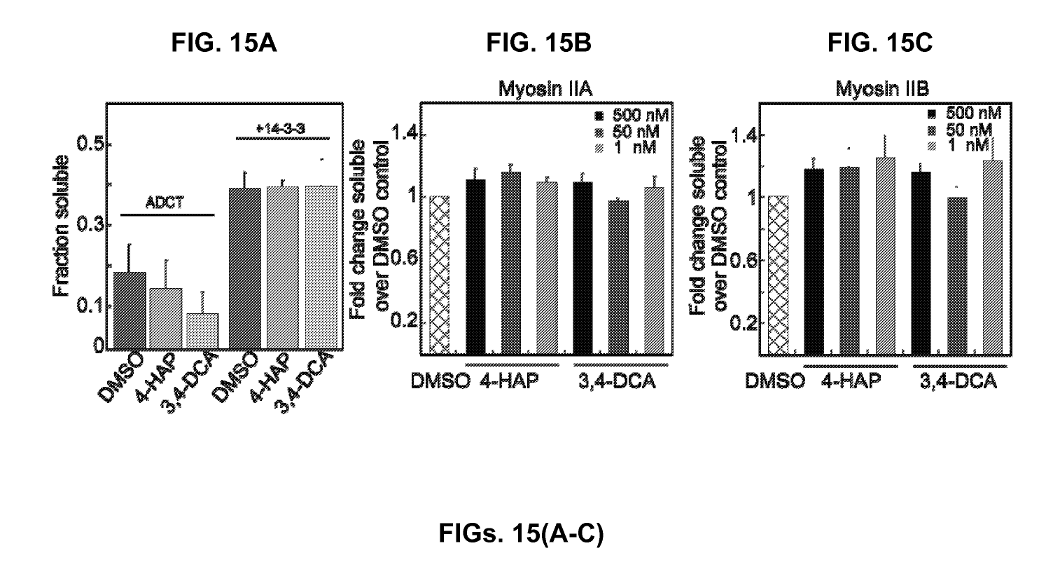

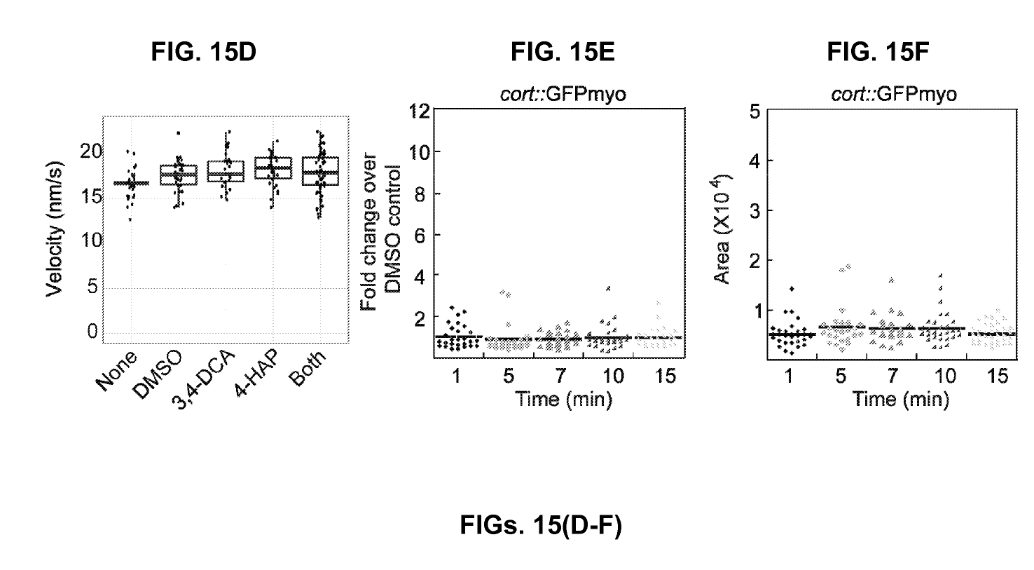

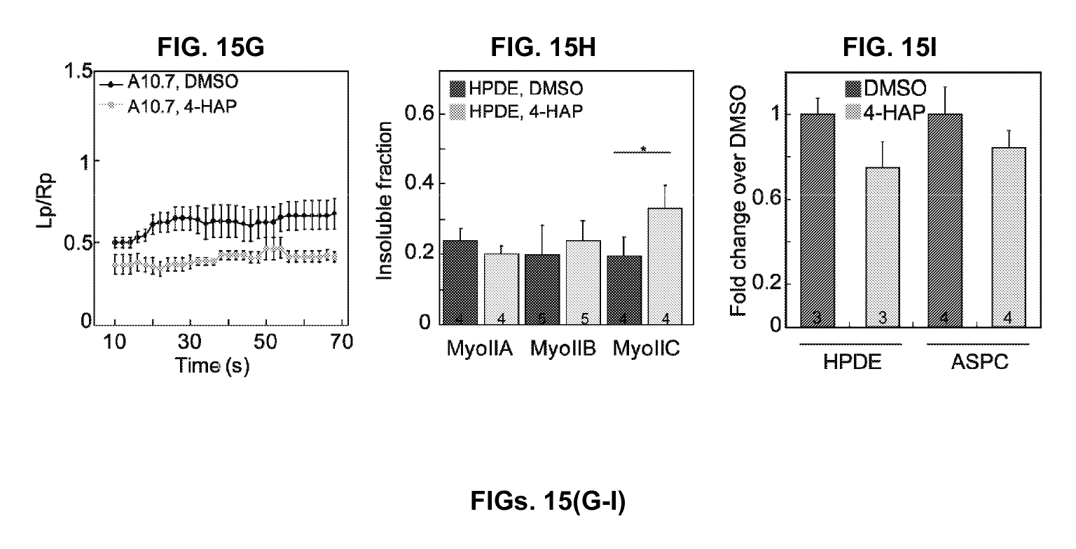

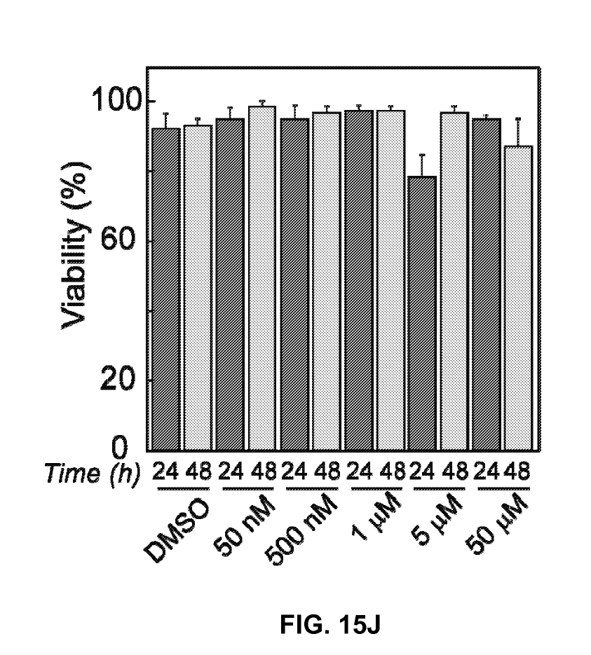

FIGS. 8(A-E) are a set of images and graphs showing 4-HAP decreases the deformability of human cells and turns the mechanical profile of pancreatic cancer cells to more WT-like mechanics, decreasing their invasive capacity. FIG. 8A: Micrographs from FIG. 8B creep tests show that 4-HAP stiffens the soft HEK293 cells (creep tests at 0.15 nN/μm²); region of aspiration, Lp; radius of pipette, Rp. FIG. 8C: Sedimentation assay shows increases in assembled myosin IIB an IIC in HEK293 cells. FIG. 8D: Similarly micrographs of aspirated cells show that 4-HAP tunes the deformability of metastatic PDAC, ASPC-1 cells. FIG. 8E: Creep tests demonstrate that the WT pancreatic cell line HPDE is stiffer than the metastatic PDAC cell-line, ASPC-1 and that 4-HAP stiffens ASPC-1 cells, shifting them towards HPDE-like mechanics (creep tests at 0.25 nN/μm²); region of aspiration, Lp; radius of pipette, Rp. FIG. 8F: 4-HAP increases assembled myosin IIC in ASPC-1 cells, and HPDE cells (FIG. 15H); n provided on bars; *p=0.04, **p=0.007, ***p=0.005. FIG. 8G: 4-HAP does not alter the cortical tension of HL-60 cells which lack the myosin IIB and IIC paralogs. All experiments presented here were performed using cell treated with 500 nM 4-HAP for 1 hr. Migration (FIG. 8H) and invasion (FIG. 8I) assays of ASPC-1 cells show a dose-dependent decrease upon 4-HAP treatment. n provided on bars; **p<0.0001, *p=0.01 for migration assay; *p=0.02 for invasion assay.

### Example 9

### Methods

CIMPAQ Work Flow

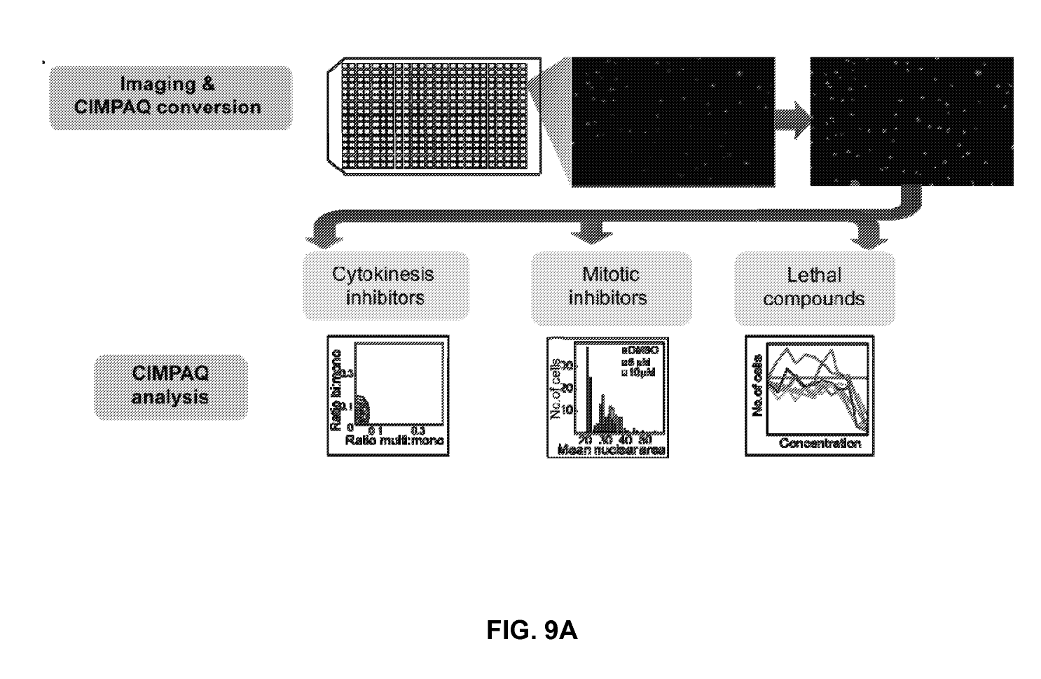

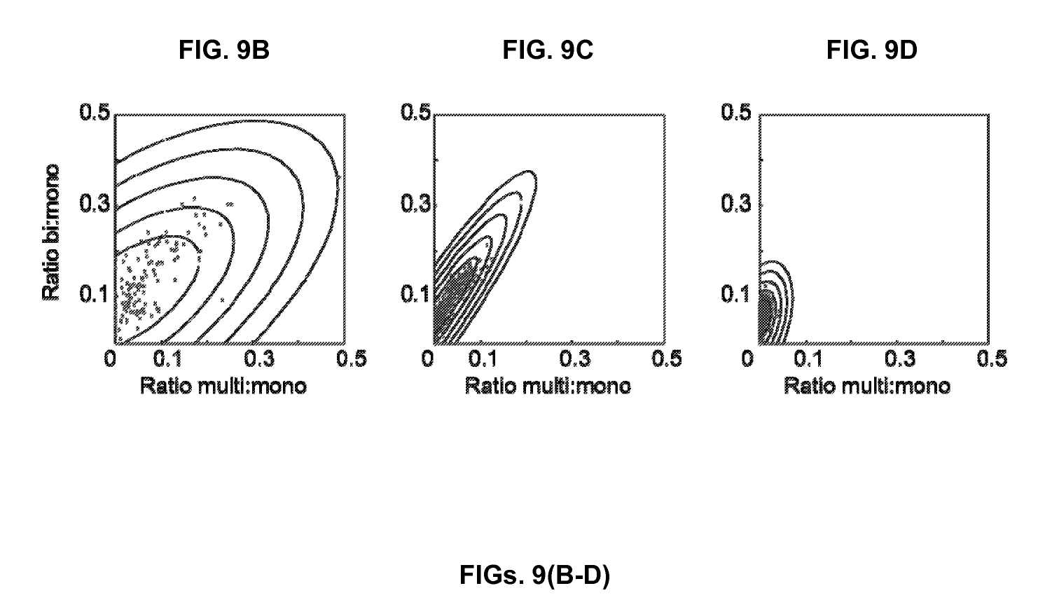

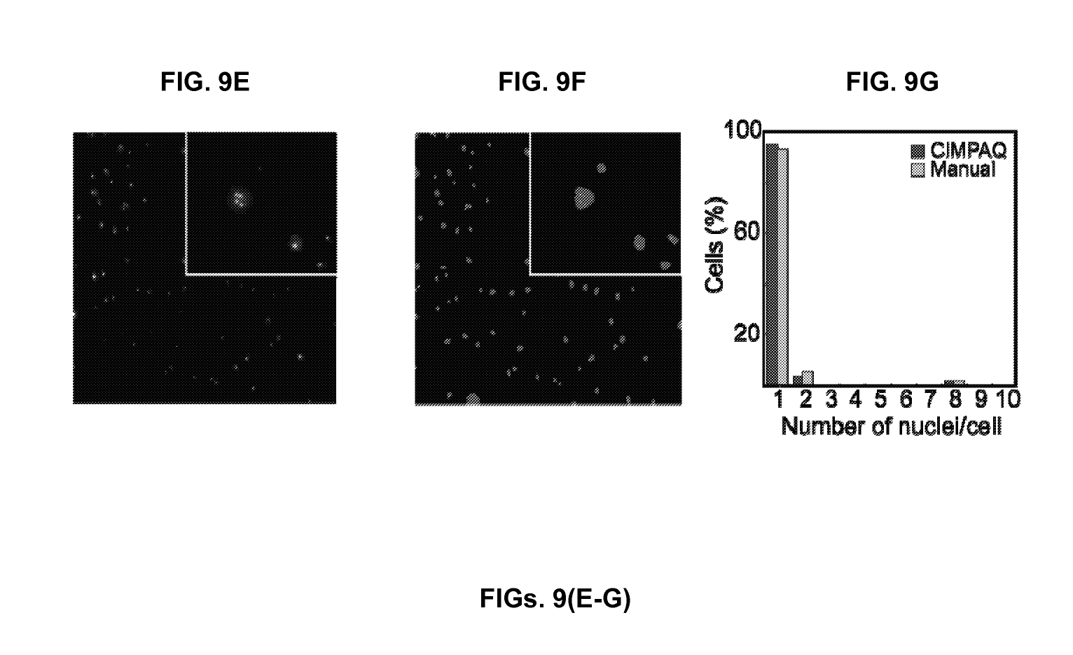

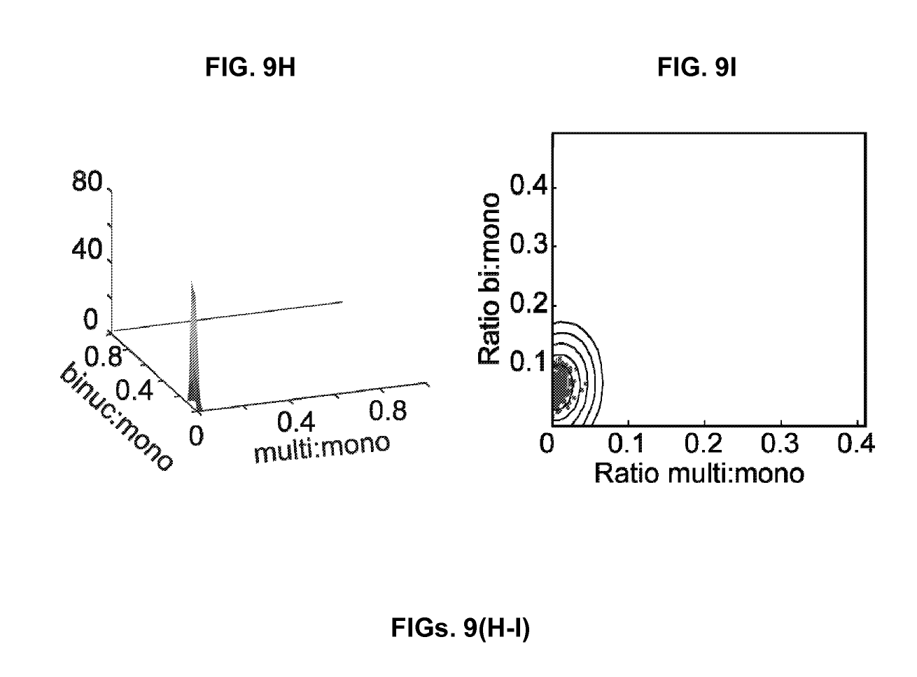

An overview of the primary screen, including CIMPAQ analysis, is presented in FIG. 9A.

Screen Development and CIMPAQ Analysis

NLS-tdTomato expressing Dictyostelium cells were challenged with 5 μM compounds from the ChemBridge Divert-SET library and imaged over three days. Raw data was segmented by CIMPAQ a designed analytical platform, which rank ordered hits based on their cytokinesis or mitotic inhibitory activity, or lethality. Hits were confirmed through a dose-dependent secondary screening.

Cell Strains and Culture

Dictyostelium discoideum strains used in this study are listed in Table 1. Dictyostelium strains were grown at 22° C. in Hans' enriched HL-5 media or ForMedium, with either G418 or hygromycin for selection. Cells grown for primary and secondary chemical screening were cultured in enriched HL-5 media (1.4XHL-5 enriched with 8% FM) with penicillin and streptomycin at 22° C. in 384-well Cyclo Olegin Polymer (COP) plates (Aurora Biotechnologies, Vancouver, British Columbia). These plates were chosen for their optical characteristics that generated a tighter distribution of nuclei/cell counts, preferable to other plates we tested [FIGS. 9(B-D)]. All other cells were cultured in ForMedium with penicillin and streptomycin at 22° C. on 10-cm Petri dishes (Robinson D N, et al., 2000) or grown in suspension in 200-ml flasks. The myoII null cells (Ruppel K M, et al., 1995), racE null cells (Gerald N, et al., 1998), cortI null cells (Robinson D N, et al., 2000), and kif12 null cells (Lakshmikanth G, et al., 2004) have been described previously. NLS-tdTomato was prepared by cloning the sequence in the pLD1 vector. Transformation of all strains was achieved by electroporation using a Genepulser-II electroporator (Bio-Rad, Hercules, Calif.).

TABLE 1
Strains used in the Application.

| | | Experimental |
|---|---|---|
| Strain | Genotype | Applications |
| WT control | Ax3 (Rep orf+) | Compound testing, MPA |
| Ax3::NLS-tdTomato | Ax3 (Rep orf+)::NLS- | Compound testing |
| | tdTomato, G418^{R}pLD1 | |
| cortI¹¹⁵¹ | cortI¹¹⁵¹ (HS1151) | CIMPAQ testing |
| racE | ΔracE | Compound testing |
| myoII | myoII (HS1) | Compound testing, MPA, |
| | | western blot |
| kif12 | kif12 (Rep orf+) | Compound testing |
| myoII::GFPmyoII; RFPtub | myoII (HS1)::GFPmyoII, | SIM, TIRF, compound |
| | G418^{R}:pBIG; RFP-α- | testing, sedimentation |
| | tubulin, Hyg^{R}:pDRH | assay, MPA |
| myoII::GFP3XAsp; RFPtub | myoII (HS1)::GFP3XAsp, | TIRF, compound testing |
| | G418^{R}:pBIG; RFP-α- | |
| | tubulin, Hyg^{R}:pDRH | |
| myoII::GFP3XAla; RFPtub | myoII (HS1)::GFP3XAla, | TIRF, compound testing |
| | G418^{R}:pBIG; RFP-α- | |
| | tubulin, Hyg^{R}:pDRH | |
| myoII::GFPS456L; RFPtub | myoII (HS1)::GFPS456L, | TIRF, compound testing |
| | G418^{R}:pBIG; RFP-α- | |
| | tubulin, Hyg^{R}:pDRH | |
| myoII::GFPS1; RFPtub | myoII (HS1)::GFPS1, | TIRF, compound testing |
| | G418^{R}:pBIG; RFP-α- | |
| | tubulin, Hyg^{R}:pDRH | |
| cortI¹¹⁵¹::GFPmyoII; RFPtub | cortI¹¹⁵¹ (HS1151):: | TIRF, compound testing |
| | GFPmyoII, G418^{R}:pBIG: | |
| | RFP-α-tubulin, Hyg^{R}:pDRH | |

Transformed cells were selected with 10-15 μg/ml G418, 15-50 μg/ml hygromycin, or both when two plasmids were transformed together. For drug treatment, cells were pre-incubated with 0.1% DMSO for 4 hrs before treatment. A10.7 cells were grown according to standard cell culture methods in DMEM high glucose (Gibco, Grand Island, N.Y.) with 1% penicillin and streptomycin and 10% FBS on cell culture petri dishes.

HPDE and ASPC-1 cells were grown according to standard cell culture methods, respectively in Keratinocyte media (Gibco, Grand Island, N.Y.), with 1% penicillin and streptomycin or RPMI 1640, L-Glutamine media (Gibco, Grand Island, N.Y.), supplemented with 1% penicillin and streptomycin, sodium pyruvate, 10% FBS and 0.2% insulin. HL-60 cells were grown in RPMI supplemented with 1% antibiotic-antimycotic mix (Invitrogen), 25 mM HEPES (Invitrogen) and 20% FBS. For drug treatment, cells were pre-incubated with 0.1% DMSO overnight. In accordance with NIH guidelines, cell lines were authenticated using short tandem repeat STR profiling in the genetic recourses core facility at Johns Hopkins University.

Micropipette Aspiration and Microscopy

Micropipette aspiration was used for cortical tension and creep response measurements. Confocal imaging was performed on a Zeiss 510 Meta with a 63× (numerical aperture [NA] 1.4) objective (Carl Zeiss, Jena, Germany). Epifluorescence and TIRF imaging was performed in a 22° C. temperature controlled room with an Olympus IX81 microscope using a 40× (NA 1.3) or 60× (NA 1.49) objective and a 1.4× optovar (Olympus, Center Valley, Pa.), as previously described. Image analysis was performed with ImageJ (rsb.info.nih.gov/ij).

In Vitro Protein Assays

The sedimentation assays were used to assess myosin II assembly in cells. The assembly assay used purified proteins (N-terminal 6×His tag, fused to the mCherry fluorophore, fused to the assembly domains of Dictyostelium myosin II (residues 1533-1823), human myosin IIA (residues 1722-1960), and human myosin IIB (residues 1729-1976), and 6×His-tagged fused Dictyostelium 14-3-3). Purified chicken nonmuscle IIB heavy meromyosin (HMM) was used for in vitro motility.

Primary and Secondary Chemical Library Screening

Ax3::NLS-tdTomato cells were plated on 384-well COP plates with a MicroFloSelect microplate dispenser (BioTek, Winooski, Vt.) at volumes of 80 μl with a cell concentration of 1000 cells/ml for the 24- and 48-hr time points and at the same volume with a cell concentration of 220 cells/ml for the 72-hr time point. Each plate contained four rows of untreated cells with 0.2% DMSO. For the remaining wells, 5 μM of each small molecule maintained at the Johns Hopkins ChemCORE facility, was added, with a final DMSO concentration of 0.2%. Almost half of the ChemBridge Divert-SET library, which is a 50,000 compound chemical diversity library, was screened over a three-day period on a Becton Dickinson Pathway 855 Bioimager System using a 20× objective (NA 0.75). Each image consisted of a montage of four images collected around the center of the well, resulting in a total size of 1344×1024 pixels per image.

Secondary chemical screening was carried out in quadruplicate, with identical culturing conditions as to the primary screen. 14 mM stocks of each compounds dissolved in 100% DMSO were diluted to the following final concentrations: 350 pM, 3.5 nM, 35 nM, 350 nM, 3.5 μM, and 35 μM.

CIMPAQ Processing, Analysis, and Hit Identification

Image Processing Using CIMPAQ:

Raw image files from both the primary and secondary screening were processed through CIMPAQ (FIGS. 9E and 9F). The single wavelength fluorescence images were converted from 16-bit format to 8-bit format. The MATLAB Image Processing Toolbox was utilized to segment the images in order to identify the nuclei and cytoplasm. The number of nuclei within each segmented cell was quantified to produce a histogram of nuclei per cell for each image (FIG. 9G). All segmented cells that were coincident with the image edge were disregarded.

Image Analysis Using CIMPAQ:

From the histogram of nuclei per cell count, the ratio of the number of multinucleate cells to the number of mononucleate cells and the ratio of the number of binucleate cells to the number of mononucleate cells were computed. Multinucleate cells are defined as cells that contain >2 nuclei. The distribution of both ratios across multiple wells were simultaneously visualized using a scatter plot, with the ratio of multinucleate cells to mononucleate cells plotted on the x-axis and the ratio of binucleate cells to mononucleate cells plotted on the y-axis (FIGS. 9H and 9I). Other information such as the average number of nuclei per cell, the mean nuclear area, and the normalized histogram with respect to total cell number were also computed.

Hit Identification Using CIMPAQ:

Compounds that generate an increase in the number of multinucleate (>2 nuclei/cell) cells are considered cytokinesis inhibitors. Because nearly all cultured cells, including Dictyostelium, have a low background (typically <5% for WT) of non-mononucleate cells, CIMPAQ spreads the data for each sample by determining the ratio (bi:mono) of binucleate (2 nuclei/cell) to mononucleate cells and the ratio (multi:mono) of multinucleate (>2 nuclei/cell) to mononucleate cells. These two ratios then define a set of Cartesian coordinates, describing the effect of each compound on a given cell-line. The coordinates for each compound are plotted on a two-dimensional graph. CIMPAQ fits the control data to a two-dimensional Gaussian distribution (FIG. 9H) and determines the contour lines for two standard deviations (2SD), 3SD, etc. from the control mean (FIG. 9I).

Hit compounds are rank-ordered based on how many SDs away they are from the untreated wells. To fit the nuclei/cell ratios, we utilized the MATLAB Statistics and Optimization Toolboxes and fitted the ratios data from the control wells with a bivariate Gaussian function. The fitted parameters of the Gaussian function were used to assign a metric number to each sample well. The metric number is defined as the value of the Gaussian function when evaluated at the ratio values computed for a sample well of interest:

metric number=f(ratio multi:mono sample, ratio bi:mono sample) where f(x,y)=fitted Gaussian function

Based on the definition, a smaller metric number corresponds to larger deviations of the ratio pair from the control mean ratios. The cutoff for a well to be considered a hit was that the ratio pair had to be >2 standard deviations from the control mean ratios. All identified hits were further categorized by the number of standard deviations away from the control mean ratios.

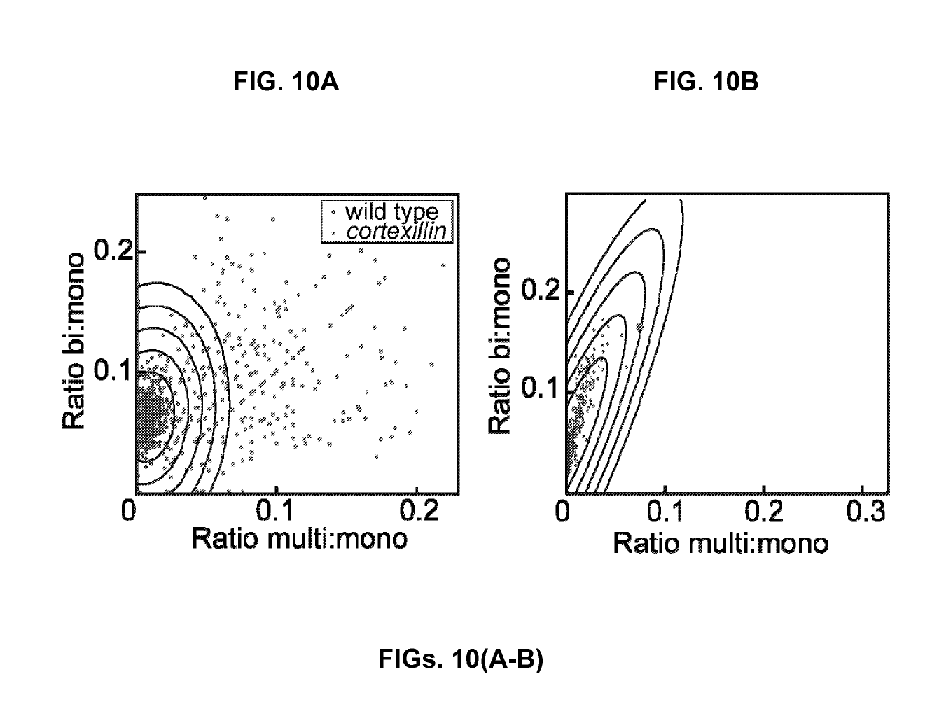

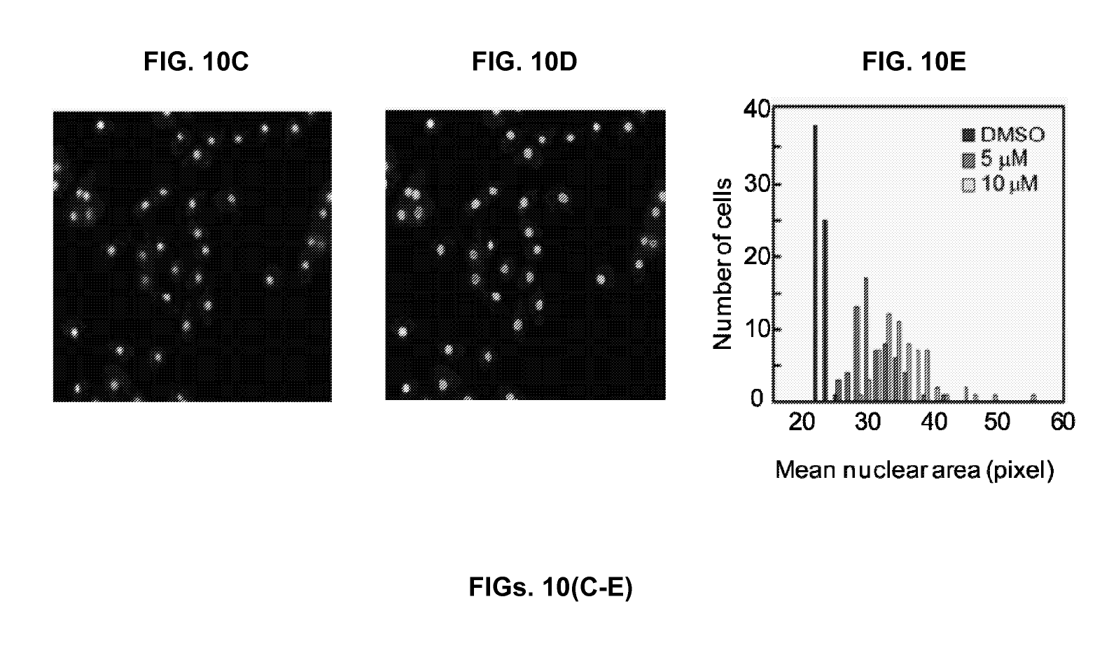

To assess the efficacy of CIMPAQ in identifying cytokinesis inhibitors, a 384-well plate containing primarily the AX3::NLS-tdTomato cell line, was randomly seeded with cortexillin I null (a cytokinesis mutant) cells transformed with the NLS-tdTomato construct. CIMPAQ was able to identify 86% of the cortexillin I-containing wells (FIG. 10A).

Mitotic Inhibitors:

Early mitotic inhibitors were identified using a simple threshold value where the average nuclear area is greater than 28 pixels. Untreated WT control cells had a tight nuclear area of 22 pixels. This threshold value reliably identified cells treated for 24 hrs and 48 hrs with 5 μM and 10 μM nocodazole, a known microtubule destabilizing agent [FIGS. 10(D-F)].

Lethal Compounds:

Lethal compounds were identified based on the total number of cells detected in the acquired image. Wells that had significantly fewer cells compared to the control (>2 SDs difference, typically 10% of average number of cells from all untreated wells) were counted as wells that contain a lethal compound at the 5 μM concentration used in the primary screening. Because data was collected over three days, growth inhibitors were also identified using similar metrics.

Library Testing of CIMPAQ:

To test CIMPAQ original pilot screens were performed on two parts of the BIOMOL collection—84 protein kinase inhibitors and 70 ion channel inhibitors (data summary of hits from these collections are listed in Tables 2 and 3; sample CIMPAQ output, FIG. 10B). In each of these setups, manual counts were compared with CIMPAQ-generated numbers. Overall, over 50,000 nuclei/cell distributions were manually counted for cross-validation of the CIMPAQ software.

TABLE 2
CIMPAQ hits identified from the kinase inhibitor collection.

| | CAS number | Name | Pathway affected |
|---|---|---|---|
| Cytokinesis | 24386-93-4 | 5-Iodotubercidin | Inhibits ERK2, adenosine kinase, |
| inhibitors (non- | | | CK1, CK2, and insulin receptor |
| lethal) | | | kinase |
| | 62004-35-7 | LFM-A13 | Tyrosine kinase inhibitor |
| Cytokinesis | 220904-83-6 | GW 5074 | A benzylidine oxindole derivative |
| inhibitors | | | that inhibits the Raf/MEK/ERK2 |
| (lethal, 5 days) | | | kinase cascade by blocking the |
| | | | kinase activity of c-Raf1 |
| | 446-72-0 | Genistein | Isoflavin that inhibits tyrosine |
| | | | kinase and has been previously |
| | | | reported to inhibit cytokinesis |
| | 63177-57-1 | Erbstatin analog | EGF receptor tyrosine kinase |
| | | | inhibitor; known IC50 (0.5 μg/ml); |
| | | | efficiently delays onset of EGF- |
| | | | induced DNA synthesis |
| | 4452-06-6 | ZM 449829 | JAK-3 tyrosine kinase inhibitor; |
| | | | binds competitively to Jak3 ATP |
| | | | site; inhibits STAT-5 |
| | | | phosphorylation and T-cell |
| | | | proliferation |
| Lethal at 5 μM | 167869-21-8 | PD-98059 | MAP kinase inhibitor |
| | 10537-47-0 | Tyrphostin 9 | PDGF receptor tyrosine kinase |
| | 172889-26-8 | PP1 | Src family tyrosine kinase inhibitor |
| | | AG-370 | PDGF receptor kinase inhibitor |
| | 548-04-9 | Hypericin | Protein kinase C inhibitor |
| Lethal at 10 μM | | 2-Hydroxy-5- | Inhibits CAM Kinase II, EGF |
| | | (2,5-dihydroxy | receptor tyrosine kinase, and pp60 |
| | | benzylamino) | kinase |
| | | benzoic acid | |
| | 6865-14-1 | Palmitoyl-DL- | PKC inhibitor |
| | | carnitine Cl | |

TABLE 3
CIMPAQ hits identified from the ion channel collection.

| | CAS | | |
|---|---|---|---|
| | number | Name | Pathway affected |
| Cytokinesis | 6151-40-2 | Quinidine | Sodium channel |
| inhibitors | | | blocker |
| (non-lethal) | 21306-56-9 | QX-314 | Sodium channel |
| | | | blocker |
| | 29094-61-9 | Glipizide | Potassium channel |
| | | | blocker |
| | 113558-89-7 | E-4031 | Potassium channel |
| | | | blocker |
| Cytokinesis | 54527-84-3 | Nicardinpin | Calcium channel |
| inhibitors | | | blocker |
| (lethal, | 2062-78-4 | Pimozide | Calcium channel |
| 5 days) | | | blocker |
| | 107254-86-4 | NPPB | Miscellaneous |
| | | | channel blocker |
| Lethal at | 52665-69-7 | Antibiotic | Intracellular |
| 5 μM | | A-23187 | calcium blocker |
| | 130495-35-1 | SKF-96365 | Calcium channel |
| | | | blocker |
| | 74764-40-2 | Bepridil | Calcium channel |
| | | | blocker |
| | 113317-61-6 | Niguldipine | Calcium channel |
| | | | blocker |

Imaging and Image Analysis

Imaging conditions during primary screen are described above. All other image analysis was performed as previously described (Kee Y S, et al., 2012). Cells were transferred from Petri dishes (with 0.1% DMSO incubation in growth media of 4 hrs) to imaging chambers and allowed to adhere for 20 min in growth media with 0.1% DMSO. After the cells adhered, the growth media was replaced with 2-(N-morpholino)ethanesulfonic acid (MES) starvation buffer (50 mM MES, pH 6.8, 2 mM MgCl₂, 0.2 mM CaCl2) with 0.1% DMSO. Confocal imaging was performed on a Zeiss 510 Meta with a 63× (numerical aperture [NA] 1.4) objective (Carl Zeiss, Jena, Germany). Epifluorescence and TIRF imaging was performed in a 22° C. temperature controlled room with an Olympus IX81 microscope using a 40× (NA 1.3) or 60× (NA 1.8) objective and a 1.4× optovar (Olympus, Center Valley, Pa.), as previously described. Image analysis was performed with ImageJ (rsb.info.nih.gov/ij). Many data sets were independently analyzed by multiple investigators.

Micropipette Aspiration Assay, Cortical Tension Measurements, and Creep Tests

The instrumental and experimental setups have been previously described (Effler J C, et al., 2006; Kee Y-S, et al., 2013). Micropipette aspiration assays were all carried out in growth media with 0.1% DMSO. For cortical tension measurements of Dictyostelium cells, pressure was applied to the cell cortex with a micropipette (2-3 μm radius, Rp) to the equilibrium pressure (AP) where the length of the cell inside the pipette (Lp) was equal to Rp. The effective cortical tension (T_{eff}) was calculated by applying the Young-Laplace equation: ΔP=2T_{eff}(1/Rp−1/Rc), where Rc is the radius of the cell and ΔP is the equilibrium pressure when Lp=Rp (Derganc J, et al., 2000; Octtaviani E, et al., 2006). For creep tests on mammalian strains, a constant aspiration stress was applied over 60 s. The micropipette radius was 3.5-4.5 μm. For quantification, the Lp/Rp ratio values was measured every two seconds and plotted as a function of time. A10.7 and HEK293 cells could only be aspirated at a low pressure range (0.15 nN/μm²), while HPDE, ASPC-1, and HL-60 cells could be aspirated at higher pressure ranges (0.25 nN/μm²) because they were stiffer.

Sedimentation Assay

Dictyostelium Sedimentation Protocol:

The sedimentation protocol was modified from Yumura et al. (Yumura S, et al., 2005) 1.5×10⁶ cells were pelleted for 5 min at 2000 rpm. The pellet was washed in MES starvation buffer (50 mM MES, pH 6.8, 0.2 M CaCl₂, 2 mM MgCl₂) and then resuspended in Buffer A (0.2 M MES, pH 6.8, 2.5 mM EGTA 5 mM MgCl₂, 0.5 mM ATP) and incubated on ice for 5 min. An equal volume of Buffer B (Buffer A+1% Triton X-100+protease inhibitor cocktail) was added, and the samples were vortexed for 5 s, followed by 5 min of incubation on ice. The supernatant, after a 10,000 g spin for 2 min at 4° C., was transferred to a fresh tube. The Triton-insoluble pellet was dissolved in 50 μl sample buffer and heated for 5 min at 100° C. 2× volume −20° C. acetone was added to the supernatant which was subsequently incubated on ice for 10 min and then centrifuged at 10000 g for 10 min at 4° C. The Triton-soluble fraction was dissolved in 50 μl sample buffer and heated for 5 min at 100° C. Samples were loaded on a 15% SDS-polyacrylamide gel.

Mammalian Cell Sedimentation Protocol:

Sedimentation protocol was adapted from the protocol above. 3×10⁶ cells were pelleted for 5 min at 2000 rpm and washed in 1 ml PBS. The pellet was resuspended in 100 μl lysis buffer (50 mM PIPES, pH 6.8, 46 mM NaCl, 2.5 mM EGTA, 1 mM MgCl₂, 1 mM ATP, 0.5% Triton X-100, and protease inhibitors—PI cocktail, PMSF, TLCK, Aprotinin). Samples were vortexed briefly and incubated on ice for 20 min, followed by centrifugation at 15,000 g for 5 min at 4° C.

Pellet was resuspended in 100 μl lysis buffer minus Triton X-100, and both pellet and supernatant fractions were heated to 100° C. for 3 min with RNaseA. Samples were incubated at 37° C. for 30 min and then heated to 100° C. in sample buffer for 5 min. Samples were loaded on a 15% SDS-polyacrylamide gel. Western blot analyses of phospho-myosin IIA was performed on whole cell lysates of cells treated as above in lysate buffer with 10 mM NaF.

Assembly Assay

Protein Purification:

Bacterial expression plasmids coding for an N-terminal 6×His tag, fused to the mCherry fluorophore, fused to the assembly domains of Dictyostelium myosin II (residues 1533-1823), human myosin IIA (residues 1722-1960), or human myosin IIB (residues 1729-1976) were generated using standard cloning techniques.

Dictyostelium 14-3-3 was also expressed in bacteria as a 6×His-tagged fusion protein (Zhou Q et al., 2010). Proteins were expressed in BL-21 Star™ (DE3) (Invitrogen) E. coli in LB shaking culture overnight at room temperature. Bacteria were harvested by centrifugation and lysed by lysozyme treatment followed by sonication, and the lysate was clarified by centrifugation. Polyethyleneimine (PEI) was added to a final concentration of 0.1% to precipitate nucleic acids, which were then removed by centrifugation. 14-3-3 precipitated in the PEI pellet, which was resuspended in column running buffer (10 mM HEPES, pH 7.1, 500 mM NaCl, 10 mM imidazole), clarified by centrifugation and filtration, and run on a Ni-NTA metal affinity column to obtain high-purity 14-3-3. The myosin constructs remained in the PEI supernatant and were precipitated by adding ammonium sulfate to 50% saturation and centrifuging. The pellet was resuspended in column running buffer and run on a Ni-NTA metal affinity column, followed by a sizing column. Protein purity was verified by SDS-PAGE followed by Coomassie Blue staining, and concentration was quantified by UV absorbance using the calculated extinction coefficient for each protein's amino acid sequence.

Assembly Assay:

In vitro assembly of myosin was conducted according to the method of Zhou et al, 2010 (Zhou Q et al., 2010), with a number of modifications. The protein concentration for each species in the tube was increased to 1 μM to ensure that the smaller protein was adequately visible by Coomassie Blue staining, and the incubation time and temperature was adjusted to 30 min at the physiological temperature for each myosin species (22° C. for Dictyostelium myosin, 37° C. for human myosins). These temperatures were also used during the centrifugation step.

Motility Assay

The chicken non-muscle IIB (NMIIB) HMM construct (residues 1-1228, GenBank™ accession number M93676, no splice insert) was purified as previously described (Norstrom M F, et al., 2010). Motility assays were performed at 22° C. and imaged on Zeiss Axiovert 200 microscope with an Andor Luca camera. The flow cells were constructed using a glass slide, two pieces of double-sided tape, and nitrocellulose-coated coverslip. Flow cells were incubated with 0.05 mg/ml green fluorescent protein antibodies (MP Biomedicals, 0.05 mg/ml in assay buffer (AB) without DTT: 25 mM KCl, 25 mM Imidazole.HCl, pH 7.5, 1 mM K.EGTA, 4 mM MgCl₂; 2 min incubation time), followed by a bovine serum albumin block (1 mg/ml in AB—as above with 10 mM DTT; 6 min incubation time). NMIIB was added to the flow cell at a concentration of 420 nM and incubated for 2 min. The flow cell was rinsed with AB and then incubated for 2 min with 50 nM F-actin in AB, stabilized with TRITC-phalloidin (American Peptide Company). The flow cell was washed again with AB. Finally, Motility Buffer was added, and actin filaments were visualized. Motility Buffer for “None” (control) contained 2 mM ATP, 2 mM free Mg²⁺, 0.086 mg/ml glucose oxidase, 0.014 mg/ml catalase, and 0.09 mg/ml glucose in AB. Motility Buffers with compounds contained 0.0036% (v/v) DMSO, and 500 nM 4-HAP, 500 nM 3,4-DCA, or 250 nM of 4-HAP and 250 nM 3,4-DCA as indicated for each experiment.

Chemistry

### Synthesis of 4-acetylphenyl (3,4-dichlorophenyl)carbamate

To a mixture of 4-hydroxyacetophenone (250 mg, 1.8 mmol) in dichloromethane (4.6 mL) at room temperature was added 3,4 dichlorophenyl isocyanate (380 mg, 2.0 mmol) in one portion, followed by addition of iPr2NEt (32 μL, 0.18 mmol) in one portion. A white precipitate formed immediately upon addition of iPr2NEt. Dichloromethane (2 mL) was added to enable more efficient stirring of the thick white mixture. The reaction was complete within 1 hr as determined by TLC analysis. The reaction mixture was partitioned between water and chloroform in a separatory funnel, and the aqueous layer was extracted with chloroform (3×10 mL). Organic layers were combined and dried over sodium sulfate. Purification of carbamate-7 was carried out on a Grace Reveleris flash chromatography system using a linear gradient (100% hexanes→100% ethyl acetate).

The carbamate product precipitated from fractions and was collected for NMR characterization. 1H NMR analysis in methanol-d4 indicated the isolated carbamate (129 mg, 20%) is identical to commercial carbamate-7 (ChemBridge) in all respects. 1H NMR (500 MHz, methanol-d4) δ 7.84-7.94 (m, 2H), 7.73 (d, J=2.04 Hz, 1H), 7.39 (d, J=8.80 Hz, 1H), 7.32 (dd, J=2.52, 8.80 Hz, 1H), 6.77-6.88 (m, 2H), 2.52 (s, 3H).

Degradation of Carbamate-7 (5180622) in DMSO:

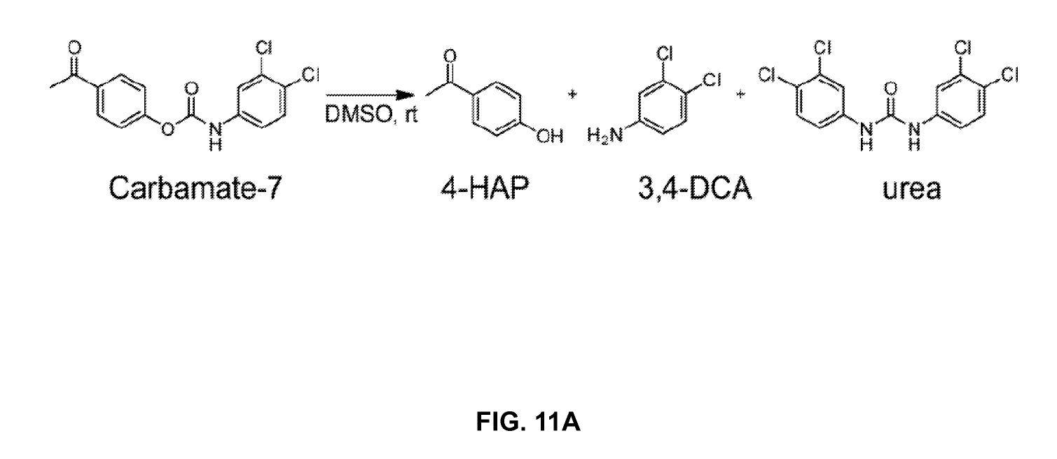

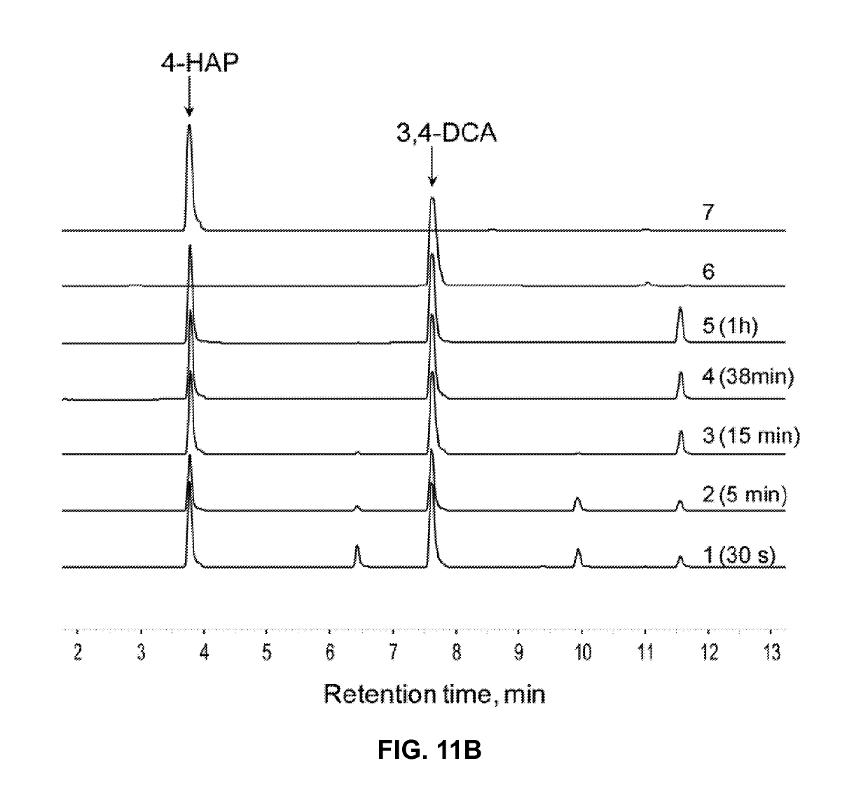

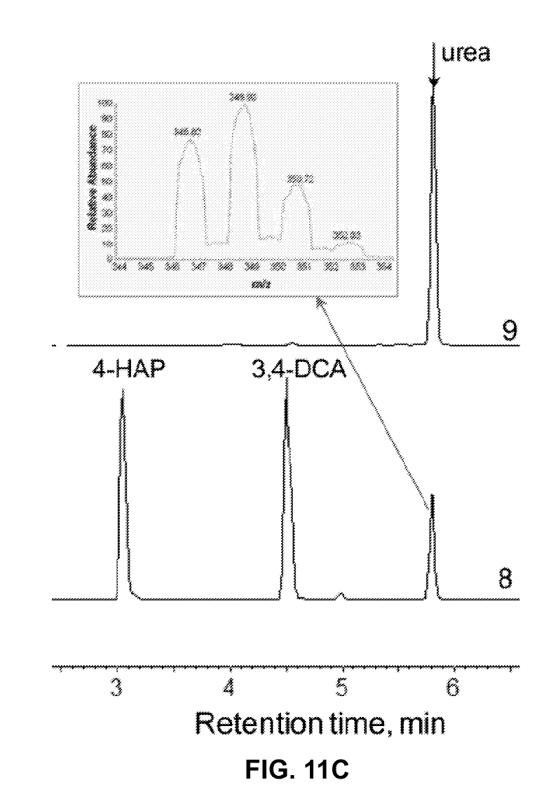

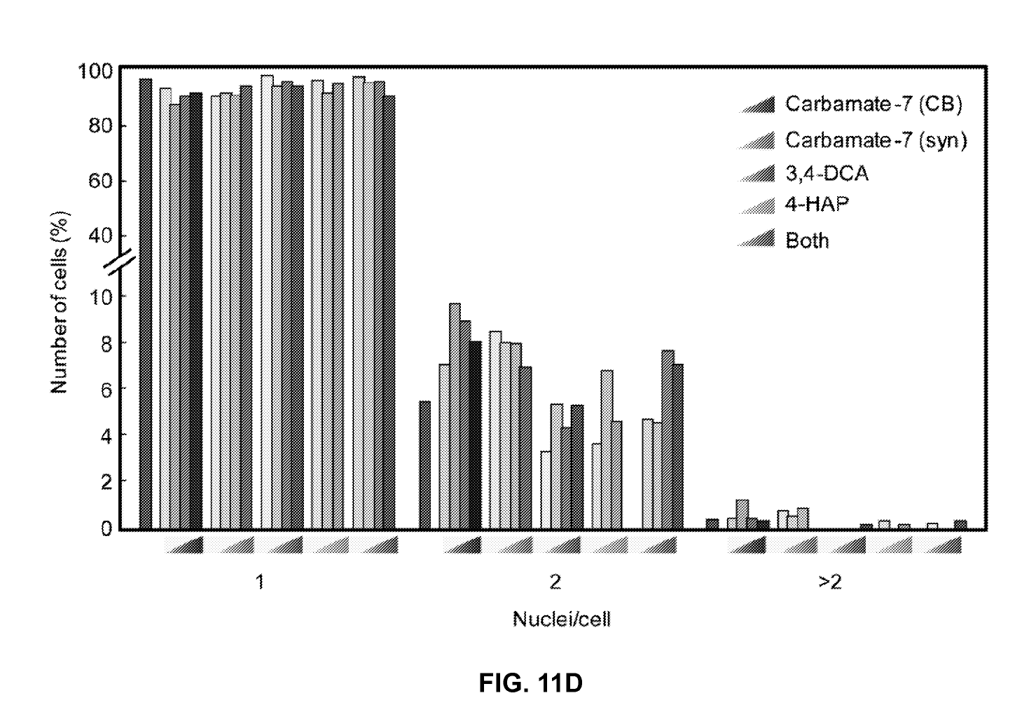

Upon standing in methanol, the product obtained above degraded within 2.5 hr, as determined by TLC analysis. Degradation appeared more rapid in DMSO, the solvent used to generate stock solutions for biological evaluation. Thus, carbamate-7 obtained either by chemical synthesis or commercially from ChemBridge was dissolved in DMSO (1 mg/mL), and a time course to study its degradation was initiated immediately upon solvation. To stop the degradation reaction such that the product distribution could be captured at early time points, aliquots (40 μL) were rapidly frozen into Eppendorf tubes incubating on dry ice. HPLC analysis on a Beckman Gold Nouveau HPLC System was performed on each sample immediately upon thawing. Carbamate-7 (5180622) degradation products were eluted at 3 mL/min from a Grace Alltima C18 column (length=53 mm, ID=7 mm, particle size=3 μM) over a linear gradient (5:95 acetonitrile/100 mM NH₄OAc (pH 6.8) to 100% 100 mM NH₄OAc (pH 6.8) over 15 min). An HPLC stack plot depicting carbamate-7 degradation over time (FIG. 11B) is displayed at 254 nm.

Synthetic and commercial carbamate-7 exhibit identical reactivity in DMSO to give 4-hydroxyacetophenone (4-HAP), 3,4-dichloroaniline (3,4-DCA) and N,N′-Bis(3,4-dichlorophenyl)urea (FIG. 11A). Comparison of the urea product to authentic N,N′-Bis(3,4-dichlorophenyl) urea was performed using a linear gradient (5:95 acetonitrile/100 mM NH₄OAc (pH 6.8) to 100% 100 mM NH₄OAc (pH 6.8) over 5 min). The urea was also confirmed by mass spectrometry analysis using a Thermo Scientific™ TSQ Vantage triple quadrupole mass spectrometer interfaced with a Dionex u3000 uHPLC. Parent mass analysis and isotopic distribution of the urea was confirmed by direct infusion for Q1 analysis in negative ion mode. Confirmation of the urea was further confirmed via characteristic fragmentation patterns determined using product ion (MS-MS) analysis monitoring in negative ion mode (FIG. 11C).

Migration Assay

Cells were starved with serum-reduced media for 24 hr, harvested from flasks with trypsin/EDTA, washed with media containing 1% FBS, and resuspended at cell density of 2-5×10⁵ cells/ml. 0.2 ml of cells were placed in the upper chamber of transwell (BD Biosciences), with 20% FBS-containing media in the lower well and incubated at 37° C. for 24 hr. Both sides of the transwell contained 4-HAP at the appropriate concentration, with final DMSO concentration at 0.0025%. The transwells were MeOH-fixed and stained with 0.5% crystal violet for 20 min, followed by counting from six random microscopic fields.

Invasion Assay

Cells were treated as in migration assay, but plated in transwells containing 2 mg/ml Matrigel (BD Biosciences).

Statistical Analyses

Data sets were collected and analyzed using KaleidaGraph (Synergy Software, Reading, Pa.). Analysis of variance (ANOVA) or Student t-tests were performed using KaleidaGraph. For all experiments, p values <0.05 were considered significant and calculated p values are included on the graphs, in the text, and/or in the figure legends.

### Example 10

### Pharmacological Activation of Myosin II to Correct Cell Mechanics Defects

Current approaches to cancer treatment focus on targeting signal transduction pathways. Here, we develop an alternative system for targeting cell mechanics for the discovery of novel therapeutics. We designed a live-cell, high-throughput chemical screen to identify mechanical modulators. We characterized 4-hydroxyacetophenone (4-HAP), which enhances the cortical localization of the mechanoenzyme myosin II, independent of myosin heavy-chain phosphorylation, thus increasing cellular cortical tension.

To shift cell mechanics, 4-HAP requires myosin II, including its full power stroke. We further demonstrated that invasive pancreatic cancer cells are more deformable than normal pancreatic ductal epithelial cells, a mechanical profile that was partially corrected with 4-HAP, which also decreased the invasion and migration of these cancer cells. Overall, 4-HAP modifies nonmuscle myosin II-based cell mechanics across phylogeny and disease states and provides proof-of-concept that cell mechanics offers a rich drug target space, allowing for possible corrective modulation of tumor cell behavior.

Carbamate-7 Affects the RacE/14-3-3/Myosin II Pathway

We developed a processing and analysis platform called Cytokinesis Image Processing Analysis Quantification (CIMPAQ), to maximize data collection from a single screen and to perform in-house data analysis. CIMPAQ allows us to analyze high content imaging data to identify cell viability, and cytokinetic and mitotic defects of Dictyostelium cells, by respectively counting cells, determining the number of nuclei per cell, and measuring the nuclear size (see FIG. 1A, and FIGS. 9E-9I, and FIG. 10 for a complete description outlining the criteria for CIMPAQ hit identification). To ensure that a full frequency distribution of all of these parameters could be extracted, each sample well contained over 400 cells per time point. This approach led to richer, more statistically relevant data sets over those normally collected for high-throughput screens. We developed and used a nuclear reporter (NLS-tdTomato) that is optimal for live cell imaging in normal growth media over multiple time points, and that allows for the number of nuclei in each cell and nuclear area to be discerned.

Proof-of-principle pilot screens were conducted (FIG. 10; Tables 1 and Table 2) and compared with manual nuclei/cell counts (FIG. 9G). Over 22,000 compounds from the ChemBridge Divert-SET library were screened using CIMPAQ. Approximately 15% of the screened compounds inhibited cell growth and 25 affected cytokinesis. Here, we focus on carbamate-7 (FIG. 2A), treatment with which resulted in an increase in the binucleate to mononucleate ratio, as well as the multinucleate to mononucleate ratio (both indicative of mild cytokinesis inhibition) at six standard deviations over untreated cells (FIG. 2B). A dose sensitivity analysis identified an increase in binucleate cells in the low nM range suggesting late mitotic or early cytokinesis failure, which became particularly evident at 48 hours (FIG. 2C).

To assess whether carbamate-7 affects known cytokinesis pathways, we targeted two spatially distinct modules—one at the equatorial plane of a dividing cell regulated by spindle signals and the mechanosensory system of myosin II/cortexillin I, and the second at the polar cortex regulated by the RacE/14-3-3/Myosin II pathway (Zhou Q et al., 2010). In a chemical-genetic epistasis analyses, we challenged mutant cell lines targeting both modules with carbamate-7. In the kinesin 6 (encoded by the kif12 locus) null cell line, cytokinesis inhibition by carbamate-7 occurred as in WT, suggesting that carbamate-7 affects a parallel cytokinesis pathway independent of the spindle signaling cascade involving kinesin 6. By contrast, carbamate-7 did not increase binucleation or multi-nucleation in myoII and racE null cell lines relative to the untreated controls. These results suggest that carbamate-7 likely works through the RacE/14-3-3/Myosin II pathway (FIG. 2D).

Epifluorescence and Structured Illumination Microscopy (SIM) studies of mCherry-racE and GFP-myosin II in their respective rescued cell lines challenged with carbamate-7 revealed no change in racE localization, but a significant increase in GFPmyosin II cortical accumulation (FIG. 3A). A dose-dependent assessment of carbamate-7 on myosin II localization using Total Internal Reflection Microscopy (TIRF) exposed an increase in the myosin II functional unit, the bipolar thick filament (BTF), at the cortex in the 500 pM range (FIGS. 3B and 3C). These results were corroborated with in vitro sedimentation assays showing an increase in the BTF-containing Triton-X-100-insoluble fraction (FIG. 3D). Because myosin II is a known effector of cell mechanics, both in Dictyostelium as well as other organisms (Zhou Q et al., 2010; Reichl E M, et al., 2007; Reichl E M, et al., 2008; Betapudi V, et al., 2006; Betapudi V, et al., 2011; Heisenberg C P, et al., 2013), we next queried whether the increase in cortical localization would impact the mechanical properties of the cell. Using micropipette aspiration (MPA) assays, we determined that acute treatment with 700 pM carbamate-7 led to a 1.4-fold increase in the cell's cortical tension (FIG. 3E), providing direct evidence that our screen successfully identified a modulator of cell mechanics.

Carbamate-7 Chemistry

The hit 5180622 (carbamate-7) was described as 4-acetylphenyl(3,4-dichlorophenyl) carbamate in the ChemBridge Divert-SET library. To validate the identity and activity of the putative carbamate-7, we synthesized and characterized an authentic sample of 4-acetylphenyl(3,4-dichlorophenyl) carbamate from 4-hydroxyacetophenone (4-HAP) and 3,4-dichlorophenyl isocyanate (FIG. 11A). Interestingly, the carbamate was unstable during purification, raising questions about its stability in the ChemBridge Divert-SET library. HPLC analysis to assess the stability of the carbamate in DMSO showed complete conversion of the carbamate to two major products, 3,4-dichloroaniline (3,4-DCA) and 4-hydroxyacetophenone (4-HAP), within 15 minutes (FIG. 11B). N,N′-Bis(3,4-dichlorophenyl)urea also appeared as a minor degradation product in DMSO. Stock solutions of carbamate-7 were subsequently analyzed and found to contain a mixture of 4-HAP, 3,4-DCA and the urea (FIG. 4A). No 4-acetylphenyl(3,4-dichlorophenyl) carbamate could be detected in the commercial stock solutions.

4-HAP Works Through Myosin II

As the degradation products arising from carbamate-7 appeared to be stable for >24 hours at 22° C., studies were carried out to determine which of these components displayed the biological activity identified above. We show with nuclei/cell distributions over a 500 pM to 5 μM concentration range that none of the degradation products alone is sufficient for cytokinesis inhibition, but that a 1:1 combination of 3,4-DCA and 4-HAP increased binucleation 2.5-fold over control cells (FIG. 4B, see FIG. 11D for full curve). We then analyzed the cortical enrichment of myosin II in cells treated with each compound and found that 4-HAP alone drives myosin II relocalization (FIGS. 4C and 4D). These results imply that we have identified a compound combination that works on two separate, yet related pathways involved in cytokinesis.

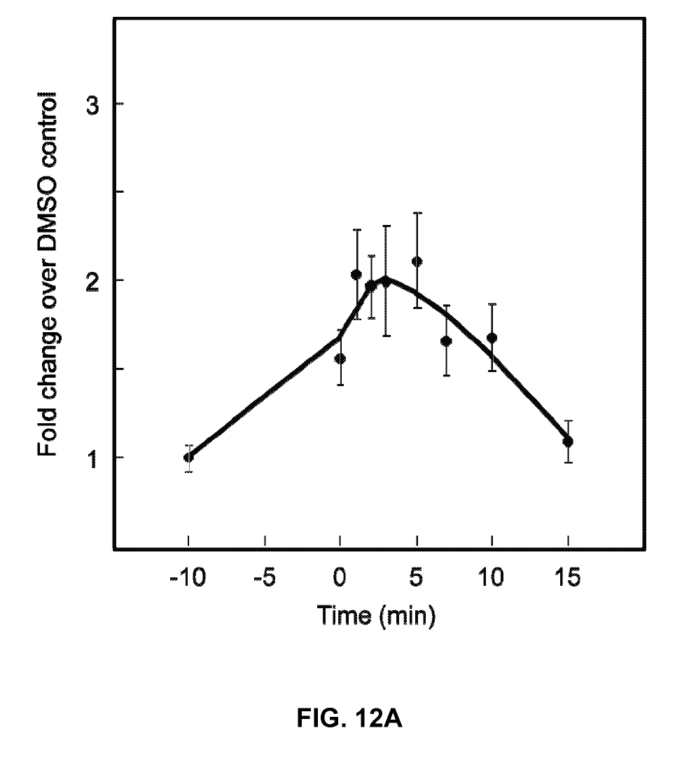

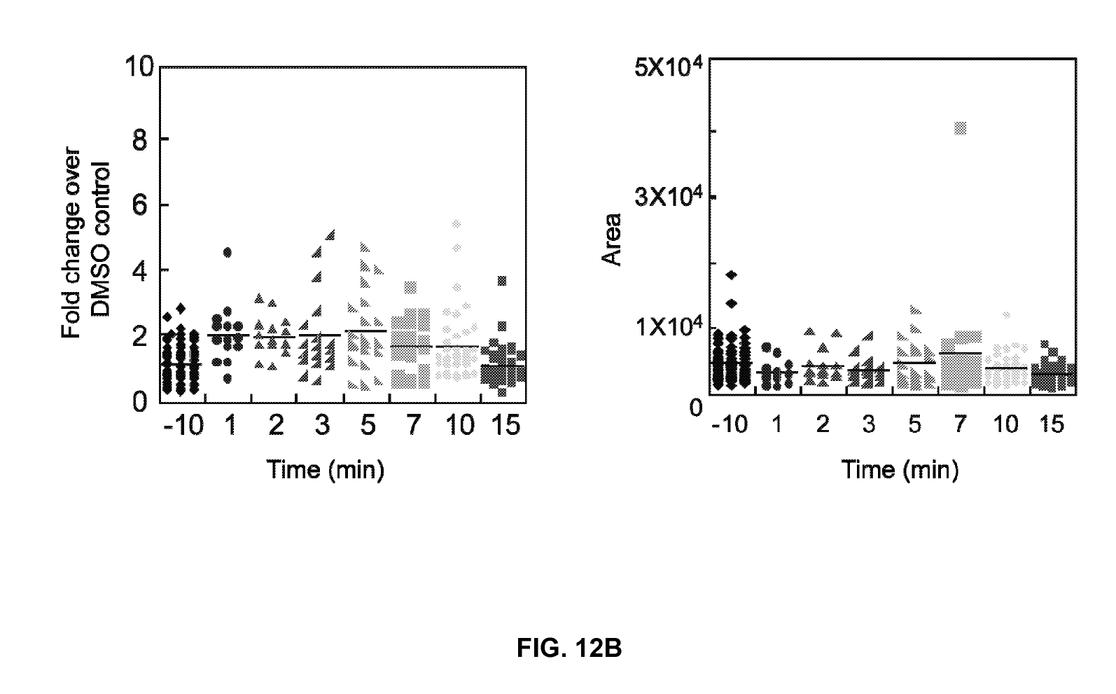

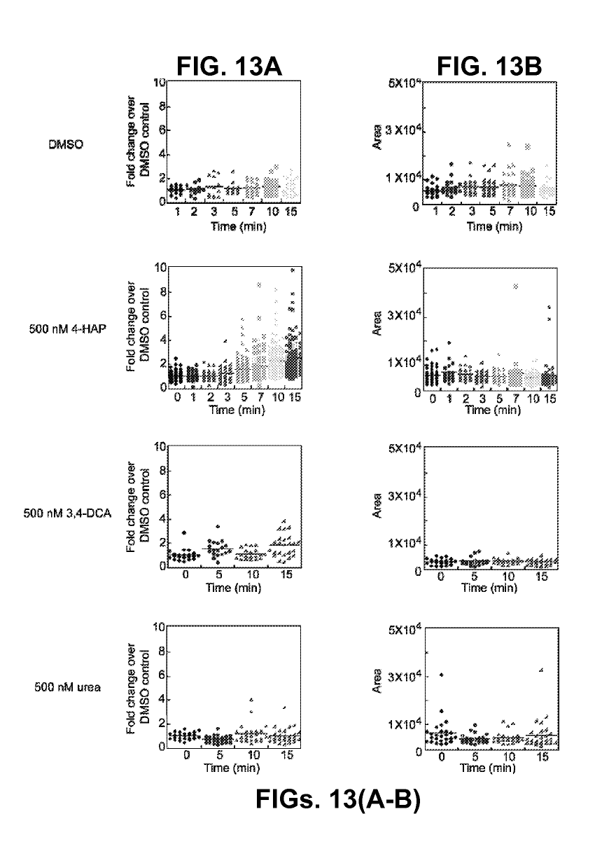

To gauge the time dependency of the myosin II cortical accumulation, we performed time-course experiments using TIRF microscopy. When challenged with 4-HAP, myosin II bipolar thick filaments accumulate at the cortex within 5 minutes, reaching steady state at 15 minutes (FIGS. 4E and 4F). In a majority of cells, the BTF structures increase in length and intensity, while in a subset of cells (˜15%) they accumulate into ribbon-like rings (FIG. 4E). This 2.5-fold increase in myosin II at the cortex is fully reversible (FIG. 12) and not the result of changes in the contact area of the cells (FIGS. 12 and 13). Neither 3,4-DCA nor the urea result in changes in myosin II cortical distribution (FIG. 13). We next asked if the 4-HAP-induced myosin II shift was responsible for the mechanical changes we previously had observed. WT cells challenged with 4-HAP displayed a 1.4-fold increase in cortical tension compared to untreated cells, while 3,4-DCA had no effect. The change in cortical tension is dependent on myosin II, as myoII null cells did not experience a similar shift in mechanics (FIG. 4G).

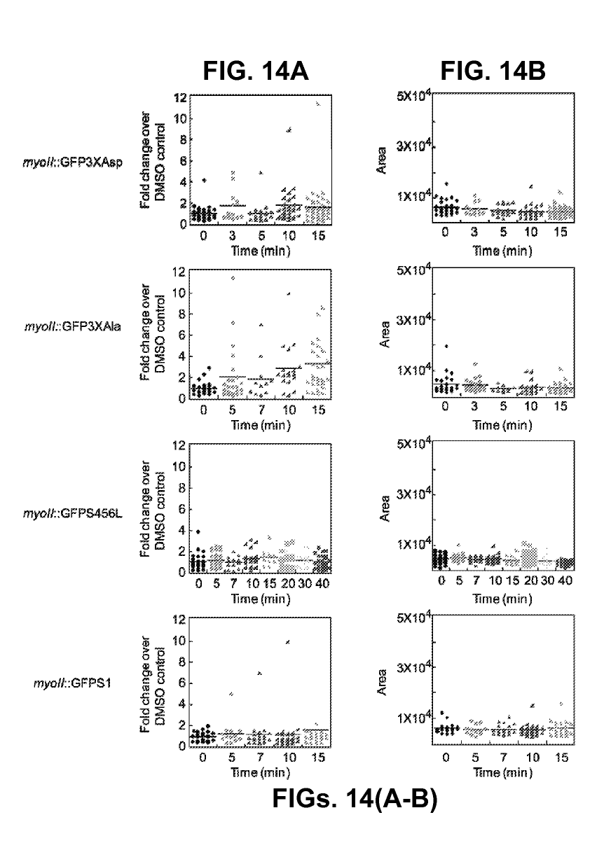

Myosin II BTF formation is regulated by the enzymatic conversion of myosin II monomers from assembly-incompetent to assembly-competent forms resulting in their dimerization and further assembly into functional BTFs (Mahajan R K, et al., 1996; Niederman R, et al., 1975). This conversion is driven by the dephosphorylation of three threonines in the myosin tail of the heavy chain, all of which are C-terminal to the assembly domain (Yumura S, et al., 2005; Egelhoff T T, et al., 1993). To determine if 4-HAP-activation of myosin II impinges on this assembly scheme, we tested the effect of 4-HAP on the in vivo assembly dynamics of the assembly-incompetent, phosphomimic form of myosin (3×Asp), as well as the assembly over-competent, unphosphorylatable form (3×Ala) in myoII null cells (Yumura S, et al., 2005; Egelhoff T T, et al., 1993; Robinson D N, et al., 2002). Both cell lines showed an increase in filament formation compared to their controls at 10 minutes post-treatment, with 3×Asp generating more short filaments, and 3×Ala increasing in filament length and intensity (FIGS. 5A and 5B; FIG. 14). To further investigate the role of the assembly domain of myosin in 4-HAP activation, we performed in vitro assembly assays on a myosin II tail fragment, assembly domain-C-terminal (ADCT), which is sufficient to reconstitute regulatable myosin II BTF assembly, as well as tail fragments from human myosin IIA and IIB. These experiments were also conducted in the presence or absence of 14-3-3, a myosin II binding partner that sequesters free myosin monomers, thus increasing the sensitivity of the assembly assay and providing a positive control for a direct effector of myosin II assembly (Zhou Q et al., 2010). In all experimental setups, 4-HAP did not affect the assembly of myosin II, including the human IIA and IIB paralogs (FIGS. 15A, 15B and 15C). These overall results imply that BTF assembly in the presence of 4-HAP is independent of myosin II heavy chain phosphorylation. Therefore, 4-HAP-induced cortical accumulation of myosin BTFs may be caused by alterations to other parts of the myosin recruitment pathway or to the myosin II ATPase cycle.

To test the latter hypothesis, we used the myosin mutant S456L. The S456L mutation disrupts the communication between the motor's ATP-binding pocket and converter domain, resulting in normal ATPase activity but a 10-fold slower actin filament sliding velocity (Murphy C T, et al., 2001). Unlike the assembly-compromised myosin mutants, myoII null cell lines complemented with GFPS456L did not show a response to 4-HAP, even when the time course was extended beyond one hour (FIGS. 5A and 5B; FIG. 14). Additionally, myoII::GFP-S456L cells did not have a change in cortical tension when treated with 4-HAP (FIG. 4G). These data highlight a highly restrictive target space for 4-HAP in the myosin II mechanochemical cycle. Further, the myosin II motor domain alone (subfragment 1-S1) of myosin II did not show an accumulation response to 4-HAP treatment, indicating that 4-HAP's effect requires dimeric myosin II or fully assembled BTFs and was not simply altering the energy state of the cell (FIGS. 5A and 5B). These results indicate that 4-HAP requires the full myosin II power stroke (FIG. 6).

We tested whether 4-HAP could affect the in vitro motility of mammalian myosin IIB and found that 4-HAP did not significantly alter this myosin's motility (FIG. 15D). However, in vitro motility assays only probe the rate-limiting step for motility under no-load conditions. In vivo, myosin II experiences load in the context of a mechanosensory control system anchored in part, by its cooperative interaction with another actin crosslinker cortexillin I (Kee Y S, et al., 2012; Ren Y, et al., 2009). If we interrupt this control system by deleting cortexillin I, 4-HAP-directed myosin II accumulation is also abolished (FIGS. 5A and 5B; FIGS. 15E and 15F). These results reveal that 4-HAP requires normal genetic pathways for myosin II accumulation to occur.

4-HAP Stiffens Pancreatic Cancer Cells and HEK293 Cells, but not HL-60 Cells

Pancreatic intraepithelial neoplasia (PanINs) that progress towards pancreatic ductal adenocarcinoma (PDAC) contain a few key genetic lesions that disproportionately affect key cytoskeletal regulators and players. For example, 95% of PDACs have early activating mutations in Kras, which modulates cell elasticity (Delpu Y, et al., 2011; Sun Q et al., 2014). Early PanINs also upregulate the actin crosslinking protein fascin, while later stages are marked by the upregulation of 14-3-3σ, a regulator of myosin II assembly (Zhou Q et al., 2010; Maitra A, et al., 2003, Clin Cancer Res; Maitra A, et al., 2003, Mod Pathol). Furthermore, serial analysis of gene expression (SAGE) of numerous pancreatic cancer cell-lines that were compared to normal pancreatic cells (HPDE) revealed alterations in the expression of several regulators of myosin II assembly and contractility (Jones S, et al., 2008). Based on these observations, we hypothesized that PDAC progression might be correlated with changes in cellular mechanics, and furthermore, that if these mechanics are myosin II-driven, they might be restored to normal, healthy mechanical profiles with 4-HAP.

To test this hypothesis, we performed MPA experiments on WT-like human pancreatic duct epithelial (HPDE) cells and two patient-derived Panc cell lines—A10.7, a liver-derived metastatic PDAC cell-line, and the commonly-used ASPC-1, an ascites-derived metastatic PDAC cell-line (Jones S, et al., 2008; Tan M H, et al., 1985). Creep tests demonstrated that these cell lines are mechanically distinct—HPDE cells are significantly stiffer than ASPC-1 or A10.7 cells. The addition of 4-HAP increased the elastic nature of both PDAC cell lines, returning them to an HPDE-like profile (FIGS. 8D and 8E, FIG. 15G). 4-HAP had a similar effect on the widely used human kidney-derived HEK293 cells (FIGS. 8A and 8B). As in Dictyostelium, 4-HAP affects myosin II assembly in human-derived cell lines: sedimentation assays showed an increase in myosin IIC BTF formation, while the myosin IIA paralog and the myosin IIA tail phosphosite (phosphor-Ser1943) showed little change (FIGS. 8C and 8F, FIG. 15I). Myosin IIB also showed a shift in assembly in response to 4-HAP, in HEK293 (FIG. 8C) and HPDE (FIG. 15H) cells, while ASPC-1 cells had no detectable myosin IIB. Due to the myosin II paralog specificity of 4-HAP in these cell lines, we next asked whether 4-HAP affects the mechanical profile of HL-60 cells, a human promyelocytic leukemia cell line which solely expresses myosin IIA. 4-HAP did not affect the cortical tension of these cells (FIG. 8G), further implying paralog specificity. 4-HAP had no dose-response effect on ASPC-1 viability (FIG. 15J).

As the initial premise of our original screen was that small molecules that modulate mechanics can affect cancer mechanobehaviors, we tested the invasive capacity of 4-HAP treated cells. ASPC-1 cells treated with 4-HAP show a dose-dependent decrease in in vitro migration and invasion (FIGS. 8H and 8I). These results suggest that the mechanical stiffening triggered by 4-HAP is sufficient to reduce the invasive capacity of metastasis-derived PDAC cells. Collectively, these results demonstrate 4-HAP's ability to alter cellular mechanics across phylogeny and disease states.

### DISCUSSION

The behavior and decision making of cells and entire tissues is derived in large part from their mechanical makeup and microenvironment. Cell mechanics define how the cell responds to its microenvironment and how it is able to display behaviors, such as tissue invasion or tumor dissemination. Myosin II has long been ascribed tremendous importance in maintaining the mechanical integrity of cells. As a mechanoenzyme, nonmuscle myosin II is pivotal in an extensive array of normal physiological mechanosensation and mechanotransduction processes, including cell division, adhesion, motility, stem cell differentiation, and tissue morphogenesis. Mutations in myosin II paralogs and myosin II regulatory proteins are associated with a number of diseases, such as the MYH9-related disease cluster (May-Hegglin Anomaly, Epstein Syndrome, and Sebastian Syndrome) (D'Apolito M, et al., 2002; Marini M, et al., 2006; Even-Ram S, et al., 2007).

Increasingly, altered non-muscle myosin II regulation is correlated with tumor progression and metastasis—the upregulation of Kras, 14-3-3, and Rac signaling leads to downregulation of contractile myosin II (Zhou Q et al., 2010; Sun Q et al., 2014; Dupont S, et al., 2011; Calvo F, et al., 2013; Liang S., et al., 2011; Schramek D., 2014; Surcel A, et al., 2010). These changes in expression, often caused by genetic lesions, can provide a mechanical differential, giving precancerous cells an advantage over their neighbors in breast and pancreatic cancer progression (Delpu Y, et al., 2011; Maitra A, et al., 2003, Clin Cancer Res; Maitra A, et al., 2003, Mod Pathol).

Affecting myosin II activity along the cellular mechanics continuum—whether through a direct disruption of myosin II-cofactor complexes or a shift in the myosin II-actin and actin-binding protein cooperative interactions that respond to mechanical stress (Luo T, et al., 2013; Luo T, et al., 2012)—has enormous therapeutic potential. Here we demonstrate the ability to identify small molecules that affect known mechanosensitive pathways by targeting the mechanical process of cell shape change that occurs during cytokinesis.

We have identified 3,4-dichloroaniline and 4-hydroxyacetophenone, the latter of which alters myosin II-dependent cell mechanics. We further demonstrate that fine-tuning myosin II dynamics can mechanically stiffen pancreatic cancer cell lines towards a more WT mechanical profile, which in turn alters the migration and invasion of these cells (FIGS. 5-6 and 8). Our strategy for identifying and characterizing small molecule modulators has broad implications not just in pancreatic adenocarcinoma, but across cancer cell types characterized by mechanical transitions, such as breast and lung cancers (Sun Q et al., 2014; Cross S E, et al., 2007).

Acetophenones, such as 4-HAP, have been previously identified as the chemical and microbial degradation products for a wide array of industrial and agricultural chemicals (Beynon K I, et al., 1973), such as bisphenol-A (BPA) (Ike M, et al., 2002) and pNP (4-(1-nonyl)phenol), where it is used for growth by some aerobic microorganisms (Vallini G, et al., 2001; Tanihata Y, et al., 2012). In addition, 4-HAP has been isolated from Cynanchum paniculatum and Cynanchum wilfordii extracts, commonly used for its anti-inflammatory and vascular-protective effects (Choi D H, et al., 2012; Choi D H, et al., 2012; Jiang Y, et al., 2011). It will be of interest to explore the possibility that 4-HAP may impact the mechanics of vascular tissue, as well as to expand upon its ability to alter myosin II dynamics in other mammalian cell types, particularly cancer cells. In addition, carbamate-7, the originally identified compound whose degradation leads to these two main byproducts, is part of a family of compounds, including propham and chlorpropham (CIPC). These compounds have been used widely in herbicides (Dolara P, et al., 1993) and were previously classified as mitotic inhibitors, with demonstrated growth defects and alterations in spindle morphology (Akashi T, et al., 1994; Hepler P K, et al., 1969; Magistrini M., et al., 1980; Oliver J M, et al., 1978; Walker G M., 1982; Clayton L., 1984). While we found that neither 3,4-DCA nor 4-HAP affected microtubule structure, we have previously demonstrated a link between microtubules and the RacE/14-3-3/MyoII pathway (Zhou Q et al., 2010). Our studies on 4-HAP and 3,4-DCA may provide further mechanistic insight into the mode of action of this class of compounds. More importantly, 4-HAP provides an important strategy for modulating cell mechanics and will be of interest to test in a wide range of disease processes, as well as in tissue engineering where cell differentiation may be guided by environmental mechanics.

### Example 11

### The Identification of 4-HAP's Mechanism of Action and Target Space

While 4-HAP's direct target remains to be identified, 4-HAP appears to work through myosin II as indicated by two key pieces of data. First, 4-HAP increases cortical tension in wild type cells, but not in myosin II null mutant cells (a complete genetic deletion) (FIG. 4G). Second, 4-HAP does not have an effect on the 5456L myosin II mutant, thus demonstrating a requirement for the full myosin II step (FIG. 5). Therefore, 4-HAP requires a full working myosin II for its effect on mechanics.

To decipher the requirements of 4-HAP on myosin II, a library of mutant myosin II proteins that affect each of the major aspects of myosin II function was used. 4-HAP's promotion of myosin II cortical localization implied a possible effect on heavy chain phosphorylation regulation of myosin II bipolar thick filament assembly. To test this hypothesis, we used the two genetic mutants that mimic the phosphorylated (3×Asp; poor assembly mutant) and non-phosphorylated (3×Ala; over-assembly mutant) states. 4-HAP still worked on these two mutants, demonstrating that its mechanism is myosin heavy chain phosphorylation-independent (FIG. 5). This result is consistent with considerable published experimental and computational work (e.g., Luo T, et al., 2013; Luo T, et al., 2012).

Having ruled out a direct involvement of heavy chain phosphorylation regulation, we turned to the motor domain. The 51 fragment (motor only) did not respond to 4-HAP. This demonstrated that there is not a global nonspecific effect such as a loss of membrane potential, which would cause the proton pump to stop producing ATP, thus leading the S1 motor to bind actin in the rigor state. Other treatments that deplete the cell of ATP also cause the S1 motor to bind to the cortex, which was not observed with 4-HAP treatment. Further, the S1 mutant data indicate that dimeric myosin II is essential for 4-HAP's effect, which is important for the mechanism of myosin II assembly.

Next, we tested the 5456L uncoupler mutant myosin II. This mutation affects an amino acid in the switch II helix, which resides inside the motor domain. This mutant residue disrupts the communication between the ATP-binding pocket and the converter domain of the motor. The consequence of this mutation is that the motor has normal ATPase activity, but uncoupled mechanochemistry. The mutant has been studied in detail for its biochemical kinetic properties and its mechanical properties (Luo T, et al., 2012; Murphy C T, et al., 2001; Reichl E M, et al., 2008; Girard K D, et al., 2006). From these studies, it is known that the 5456L myosin has two defects: a short 2-nm step size, which is ¼ of the WT 8-nm step, and a 3-fold longer ADP-bound state than WT myosin II. Because the velocity of a motor is dependent on the step size divided by the strong actin-bound state time (generally dominated by the ADP-bound state under no-force conditions), this motor slides actin filaments at 1/10 (1/(4×3)) of the WT velocity. The 5456L mutant is insensitive to 4-HAP (FIG. 5).

This observation is enormously restrictive for what the cellular mechanism of 4-HAP can be. To explain why, we start with a molecular view of what the motor is doing. To begin, ATP binds the myosin II motor, which causes the motor to release from the actin filament. The motor rapidly hydrolyzes the ATP to ADP.Pi, and it is not until the motor encounters an actin filament that it releases the Pi. Upon encountering an actin filament, the motor binds weakly, then tightly as the Pi is released (see FIG. 6 for cartoon). This all happens normally in 5456L, which is why its Vmax of ATP hydrolysis is normal. The WT and 5456L motors undergo a ˜2 nm step, at which point they have reached the isometric state. Here, WT and 5456L diverge in what they do. WT extends the power stroke another 6 nm, to complete the full 8 nm step. Consequently, this larger step will lead to a bigger deformation in any compliant elements throughout the motor or bipolar thick filament. However, 5456L exits the normal pathway where it does not take any larger step, waits a little longer before letting go of the ADP, ultimately rebinds ATP and releases from the actin filament. Thus, the S456L mutant identifies a very specific place in the myosin II mechanochemical cycle that 4-HAP depends on for its ability to promote myosin II accumulation.

Moving up to the cortical actin network and whole cell, it is now important to consider how S456L works at these hierarchical levels. At the cellular level, S456L acts as though it is an inert, dead myosin II in the context of interphase cells that are not experiencing mechanical stress (Reichl E M, et al., 2008; Girard K D, et al., 2006). However, as soon as a mechanical stress propagates through the network, S456L behaves as though it is a WT myosin motor.

This WT behavior is seen in two scenarios: cytokinesis furrow ingression (Reichl E M, et al., 2008) and when mechanical stress is imposed using aspiration (Ren Y, et al., 2009; Luo T, et al., 2013). Thus, physiological (cytokinesis) and imposed (aspiration) mechanical stresses rescue the activity of this mutant motor. Because S456L can accumulate in response to mechanical stress, it implies that it can sample the isometric, cooperative binding state (Luo T, et al., 2012). Importantly, the force-dependent bond length of WT myosin II is ˜1-2 nm, which is similar to S456L's 2 nm step. Thus, 4-HAP must do something that depends on the remaining 6 nm of the WT step. We currently suspect 4-HAP helps stabilize directly or indirectly the stretching of another compliant element in the myosin II tail, which assists in another aspect of thick filament assembly. Applied mechanical stresses are able to stretch this element even if the motor cannot exert enough deformation (S456L short step-size) so long as the motor can enter the cooperative binding state. 4-HAP may then affect this cross-talk between the motor and the tail.

Finally, myosin II accumulation occurs as a result of the function of a control system constructed by two feedback loops (Kee Y S, et al., 2012). The implication is that myosin II cortical accumulation depends on multiple signal inputs, which include biochemical and mechanical signaling that are integrated. If we break this control system at a key point by deleting cortexillin I—a specific membrane anchoring-actin crosslinking protein, which cooperates with myosin II for accumulation in response to mechanical stress (Kee Y S, et al., 2012; Ren Y, et al., 2009; Luo T, et al., 2013)—we also block myosin II accumulation by 4-HAP (FIGS. 5A and 5B; Fig. S9E, F). This result demonstrates that 4-HAP requires an intact control system for myosin II accumulation. If the 4-HAP-directed myosin II accumulation were non-specific, one might expect that the accumulation would be independent of specific known pathways that the cell uses for myosin II accumulation during normal processes like cytokinesis.

The present invention has been described in terms of one or more preferred embodiments, and it should be appreciated that many equivalents, alternatives, variations, and modifications, aside from those expressly stated, are possible and within the scope of the invention.

### REFERENCES

1. Moser, T. L., Kenan, D. J., Ashley, T. A., Roy, J. A., Goodman, M. D., Misra, U. K., Cheek, D. J., and Pizzo, S. V. (2001). Endothelial cell surface F1-F0 ATP synthase is active in ATP synthesis and is inhibited by angiostatin. Proc Natl Acad Sci USA 98, 6656-6661.
2. Malik, F. I., Hartman, J. J., Elias, K. A., Morgan, B. P., Rodriguez, H., Brejc, K., Anderson, R. L., Sueoka, S. H., Lee, K. H., Finer, J. T., et al. (2011). Cardiac myosin activation: a potential therapeutic approach for systolic heart failure. Science 331, 1439-1443.
3. Straight, A. F., Cheung, A., Limouze, J., Chen, I., Westwood, N. J., Sellers, J. R., and Mitchison, T. J. (2003). Dissecting temporal and spatial control of cytokinesis with a myosin II inhibitor. Science 299, 1743-1747.
4. Ostap, E. M. (2002). 2,3-Butanedione monoxime (BDM) as a myosin inhibitor. J Muscle Res Cell Motil 23, 305-308.
5. Ishihara, H., Martin, B. L., Brautigan, D. L., Karaki, H., Ozaki, H., Kato, Y., Fusetani, N., Watabe, S., Hashimoto, K., Uemura, D., et al. (1989). Calyculin A and okadaic acid: inhibitors of protein phosphatase activity. Biochem Biophys Res Commun 159, 871-877.
6. Ishihara, H., Ozaki, H., Sato, K., Hori, M., Karaki, H., Watabe, S., Kato, Y., Fusetani, N., Hashimoto, K., Uemura, D., et al. (1989). Calcium-independent activation of contractile apparatus in smooth muscle by calyculin-A. J Pharmacol Exp Ther 250, 388-396.
7. Makishima, M., Honma, Y., Hozumi, M., Sampi, K., Hattori, M., and Motoyoshi, K. (1991). Induction of differentiation of human leukemia cells by inhibitors of myosin light chain kinase. FEBS Lett 287, 175-177.
8. Saitoh, M., Ishikawa, T., Matsushima, S., Naka, M., and Hidaka, H. (1987). Selective inhibition of catalytic activity of smooth muscle myosin light chain kinase. J Biol Chem 262, 7796-7801.
9. Uehata, M., Ishizaki, T., Satoh, H., Ono, T., Kawahara, T., Morishita, T., Tamakawa, H., Yamagami, K., Inui, J., Maekawa, M., et al. (1997). Calcium sensitization of smooth muscle mediated by a Rho-associated protein kinase in hypertension. Nature 389, 990-994.
10. Luo T, et al. (2012) Understanding the cooperative interaction between myosin II andactin cross-linkers mediated by actin filaments during mechanosensation. Biophys. 1.102(2):238-247.
11. Murphy C T, Rock R S, & Spudich J A (2001) A myosin II mutation uncouples ATPase activity from motility and shortens step size. Nat. Cell Biol. 3:311-315.
12 Reichl E M, et al. (2008) Interactions between myosin and actin crosslinkers control cytokinesis contractility dynamics and mechanics. Curr. Biol. 18(7):471-480.
13. Girard K D, Kuo S C, & Robinson D N (2006) Dictyostelium myosin II mechanochemistry promotes active behavior of the cortex on long time scales. Proc Natl Acad Sci USA 103(7):2103-2108.
14. Ren Y, et al. (2009) Mechanosensing through cooperative interactions between myosin II and the actin crosslinker cortexillin. Curr Biol 19(17):1421-1428.
15. Luo T, Mohan K, Iglesias P A, & Robinson D N (2013) Molecular mechanisms of cellular mechanosensing. Nat. Mater. 12:1064-1071.
16. Kee Y S, et al. (2012) A mechanosensory system governs myosin II accumulation in dividing cells. Mol Biol Cell 23:1510-1523.
17. Robinson D N & Spudich J A (2000) Dynacortin, a genetic link between equatorial contractility and global shape control discovered by library complementation of a Dictyostelium discoideum cytokinesis mutant. J. Cell Biol. 150(4):823-838.
18. Ruppel K M & Spudich J A (1995) Myosin motor function: structural and mutagenic approaches. Curr Opin Cell Biol 7(1):89-93.
19. Gerald N, Dai J, Ting-Beall H P, & De Lozanne A (1998) A role for Dictyostelium racE in cortical tension and cleavage furrow progression. J Cell Biol 141(2):483-492.
20. Lakshmikanth G, Warrick H M, & Spudich J A (2004) A mitotic kinesin-like protein required for normal karyokinesis, myosin localization to the furrow, and cytokinesis in Dictyostelium. Proc. Natl. Acad. Sci. USA 101(47):16519-16524.
21. Effler J C, et al. (2006) Mitosis-specific mechanosensing and contractile-protein redistribution control cell shape. Curr Biol 16(19):1962-1967.
22. Kee Y-S & Robinson D N (2013) Micropipette Aspiration for Studying Cellular Mechanosensory Responses and Mechanics. Dictyostelium Protocols II: Methods Mol. Biol. 983:367-382.
23. Derganc J, Bozic B, Svetina S, & Zeks B (2000) Stability analysis of micropipette aspiration of neutrophils. Biophys J 79(1):153-162.
24. Octtaviani E, Effler J C, & Robinson D N (2006) Enlazin, a natural fusion of two classes of canonical cytoskeletal proteins, contributes to cytokinesis dynamics. Mol. Biol. Cell 17(12):5275-5286.
25. Yumura S, et al. (2005) Multiple myosin II heavy chain kinases: roles in filament assembly control and proper cytokinesis in Dictyostelium. Mol. Biol. Cell 16(9):4256-4266.
26. Zhou Q et al. (2010) 14-3-3 coordinates microtubules, Rac, and myosin II to control cell mechanics and cytokinesis. Curr Biol 20(21):1881-1889.
27. Norstrom M F, Smithback P A, & Rock R S (2010) Unconventional processive mechanics of non-muscle myosin JIB. J. Biol Chem 285(34):26326-26334.
28. Reichl E M & Robinson D N (2007) Putting the brakes on cytokinesis with alpha-actinin. Dev. Cell 13:460-462.
29. Betapudi V, Licate L S, & Egelhoff T T (2006) Distinct roles of nonmuscle myosin II isoforms in the regulation of MDA-MB-231 breast cancer cell spreading and migration. Cancer Res 66(9):4725-4733.
30. Betapudi V, Gokulrangan G, Chance M R, & Egelhoff T T (2011) A proteomic study of myosin II motor proteins during tumor cell migration. J Mol Biol 407(5):673-686.
31. Heisenberg C P & Bellaiche Y (2013) Forces in tissue morphogenesis and patterning. Cell 153(5):948-962.
32. Mahajan R K & Pardee J D (1996) Assembly mechanism of Dictyostelium myosin II: Regulation by K+, Mg2+, and actin filaments. Biochemistry 35:15504-15514.
33. Niederman R & Pollard T D (1975) Human platelet myosin. II. In vitro assembly and structure of myosin filaments. J. Cell Biol. 67:72-92.
34. Egelhoff T T, Lee R J, & Spudich J A (1993) Dictyostelium myosin heavy chain phosphorylation sites regulate myosin filament assembly and localization in vivo. Cell 75:363-371.
35. Robinson D N, Cavet G, Warrick H M, & Spudich J A (2002) Quantitation of the distribution and flux of myosin-II during cytokinesis. BMC Cell Biol 3:4.
36. Delpu Y, et al. (2011) Genetic and epigenetic alterations in pancreatic carcinogenesis. Curr Genomics 12(1):15-24.
37. Sun Q et al. (2014) Competition between human cells by entosis. Cell Res. 24(11):1299-1310.
38. Maitra A, et al. (2003) Global expression analysis of well-differentiated pancreatic endocrine neoplasms using oligonucleotide microarrays. Clin Cancer Res 9(16 Pt 1):5988-5995.
39. Maitra A, et al. (2003) Multicomponent analysis of the pancreatic adenocarcinoma progression model using a pancreatic intraepithelial neoplasia tissue microarray. Mod Pathol 16(9):902-912.
40. Tan M H & Chu T M (1985) Characterization of the tumorigenic and metastatic properties of a human pancreatic tumor cell line (AsPC-1) implanted orthotopically into nude mice. Tumour Biol 6(1):89-98.
41. D'Apolito M, Guarnieri V, Boncristiano M, Zelante L, & Savoia A (2002) Cloning of the murine non-muscle myosin heavy chain IIA gene ortholog of human MYH9 responsible for May-Hegglin, Sebastian, Fechtner, and Epstein syndromes. Gene 286(2):215-222.
42. Jones S, et al. (2008) Core signaling pathways in human pancreatic cancers revealed by global genomic analyses. Science 321(5897):1801-1806.
43. Marini M, et al. (2006) Non-muscle myosin heavy chain IIA and IIB interact and co-localize in living cells: relevance for MYH9-related disease. Intl Mol Med 17(5):729-736.
44. Even-Ram S & Yamada K M (2007) Of mice and men: Relevance of cellular and molecular characterizations of myosin IIA to MYH9-related human disease. Cell Adh Migr 1(3):152-155.
45. Dupont S, et al. (2011) Role of YAP/TAZ in mechanotransduction. Nature 474(7350):179-183.
46. Calvo F, et al. (2013) Mechanotransduction and YAP-dependent matrix remodelling is required for the generation and maintenance of cancer-associated fibroblasts. Nat Cell Biol 15 (6):637-646.
47. Liang S, et al. (2011) MicroRNA let-7f inhibits tumor invasion and metastasis by targeting MYH9 in human gastric cancer. PLoS One 6(4):e18409.
48. Schramek D, et al. (2014) Direct in vivo RNAi screen unveils myosin IIa as a tumor suppressor of squamous cell carcinomas. Science 343(6168):309-313.
49. Surcel A, Kee Y S, Luo T, & Robinson D N (2010) Cytokinesis through biochemicalmechanical feedback loops. Semin Cell Dev Biol 21(9):866-873.
50. Cross S E, Jin Y S, Rao J, & Gimzewski J K (2007) Nanomechanical analysis of cells from cancer patients. Nat Nanotechnol 2(12):780-783.
51. Vallini G, Frassinetti S, D'Andrea F, Catelani G, & Agnolucci M (2001) Biodegredation of 4-(1-nonyl)phenol by axenic cultures of the yeast Candida aquaetextoris: identification of microbial breakdown products and proposal of a possible metabolic pathway. Int. Biodeter. Biodegr. 47:133-140.
52. Tanihata Y, Watanabe M, Mitsukura K, & Maruyama K (Oxidative degradation of 4-hydroxyacetophenone in Arthrobacter sp. TGJ4. Biosci Biotechnol Biochem 76(4):838-840.
53. Choi D H, Lee Y J, Kim J S, Kang D G, & Lee H S (2012) Cynanchum wilfordii ameliorates hypertension and endothelial dysfunction in rats fed with high fat/cholesterol diets. Immunopharmacol Immunotoxicol 34(1):4-11.
54. Choi D H, et al. (2012) Improved endothelial dysfunction by Cynanchum wilfordii in apolipoprotein E(−/−) mice fed a high fat/cholesterol diet. J Med Food 15(2):169-179.
55. Jiang Y, et al. (2011) Chemical Metabolites of Cynanchum wilfordii and the chemotaxonomy of two species of the family Asclepiadacease, C. wilfordii and C. auriculatum. Arch Pharm Res 34(12):2021-2027.
56. Dolara P, Vezzani A, Caderni G, Coppi C, & Torricelli F (1993) Genetic toxicity of a mixture of fifteen pesticides commonly found in the Italian diet. Cell Biol Toxicol 9(4):333-343.
57. Akashi T, Kanbe T, & Tanaka K (1994) The role of the cytoskeleton in the polarized growth of the germ tube in Candida albicans. Microbiology 140 (Pt 2):271-280.
58. Hepler P K & Jackson W T (1969) Isopropyl N-phenylcarbamate affects spindle microtubule orientation in dividing endosperm cells of Haemanthus katherinae Baker. J Cell Sci 5(3):727-743.
59. Magistrini M & Szollosi D (1980) Effects of cold and of isopropyl-N-phenylcarbamate on the second meiotic spindle of mouse oocytes. Eur J Cell Biol 22 (2):699-707.
60. Oliver J M, Krawiec J A, & Berlin R D (1978) A carbamate herbicide causes microtubule and microfilament disruption and nuclear fragmentation in fibroblasts. Exp Cell Res 116(1):229-237.
61. Walker G M (1982) Cell cycle specificity of certain antimicrotubular drugs in Schizosaccharomyces pombe. J Gen Microbiol 128(1):61-71.
62. Clayton L & Lloyd C W (1984) The relationship between the division plane and spindle geometry in Allium cells treated with CIPC and griseofulvin: an anti-tubulin study. Eur J Cell Biol 34(2):248-253.
63. Girdler F, et al. (2006) Validating Aurora B as an anti-cancer drug target. J. Cell Sci. 119:3664-3675.
64. de Weger V A, Beijnen J H, & Schellens J H (2014) Cellular and clinical pharmacology of the taxanes docetaxel and paclitaxel—a review. Anticancer Drugs.
65. Discher D E, Janmey P, & Wang Y L (2005) Tissue cells feel and respond to the stiffness of their substrate. Science 310(5751):1139-1143.
66. Bhadriraju K & Hansen L K (2002) Extracellular matrix- and cytoskeleton-dependent changes in cell shape and stiffness. Exp Cell Res 278(1):92-100.
67. Chiang A C & Massague J (2008) Molecular basis of metastasis. N Engl J Med 359(26):2814-2823.
68. Wakatsuki T, Schwab B, Thompson N C, & Elson E L (2001) Effects of cytochalasin D and latrunculin B on mechanical properties of cells. J Cell Sci 114(Pt 5):1025-1036.

We claim:

1. A method for modulating cell mechanics of a disease condition in a subject comprising the step of administering an effective amount of compound (I) or its derivatives or a mixture of their constituents, where the compound has the formula: 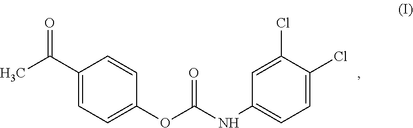 [SMILES: CC(=O)c1ccc(OC(=O)Nc2ccc(Cl)c(Cl)c2)cc1]
wherein myosin II is activated, cell mechanics are modulated and the disease condition is treated in the subject.

2. The method of claim 1, wherein the method of administering is systemic delivery selected from the group consisting of oral, parenteral, intranasal, sublingual, rectal, and transdermal administration.

3. A method for modulating cell mechanics of a disease condition in a subject comprising the step of administering an effective amount of a compound (II) or its derivatives or a mixture of their constituents, where the compound has the formula: 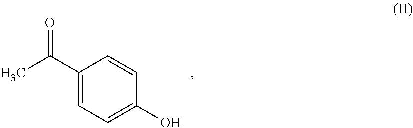
wherein myosin II is activated, cell mechanics are modulated and the disease condition is treated in the subject.

4. The method of claim 3, wherein the method of administering is systemic delivery selected from the group consisting of oral, parenteral, intranasal, sublingual, rectal, and transdermal administration.

5. The method of claim 3, wherein the method further comprises the step of administering an effective amount of a compound having the formula:

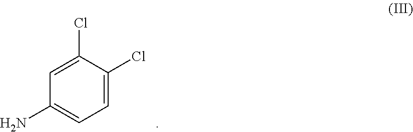

6. The method of claim 5, wherein the method of administering is systemic delivery selected from the group consisting of oral, parenteral, intranasal, sublingual, rectal, and transdermal administration.

7. A method for modulating cell mechanics of a disease condition in a subject comprising the step of administering an effective amount of compound (IV) or its derivatives or a mixture of their constituents, wherein the compound has the formula: 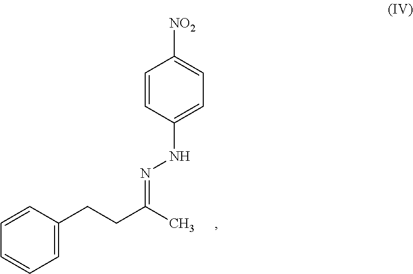 [SMILES: C/C(CCc1ccccc1)=N\Nc1ccc([N+](=O)[O-])cc1]
wherein cytokinesis is modulated and the disease condition is treated in the subject.

8. The method of claim 7, wherein the method of administering is systemic delivery selected from the group consisting of oral, parenteral, intranasal, sublingual, rectal, and transdermal administration.

9. A compound having the formula: 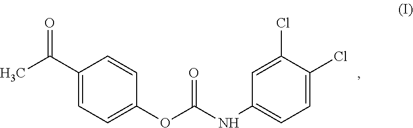 [SMILES: CC(=O)c1ccc(OC(=O)Nc2ccc(Cl)c(Cl)c2)cc1]
for use in activating myosin II to treat a disease condition in a subject.

10. The compound of claim 9, wherein the compound is administered by systemic delivery selected from the group consisting of oral, parenteral, intranasal, sublingual, rectal, and transdermal administration.

11. A compound having the formula: 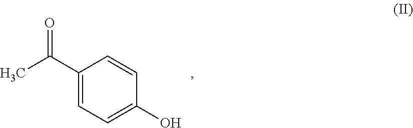
for use in activating myosin II to treat a disease condition in a subject.

12. The compound of claim 11, wherein the compound is administered by systemic delivery selected from the group consisting of oral, parenteral, intranasal, sublingual, rectal, and transdermal administration.

13. A compound having the formula: 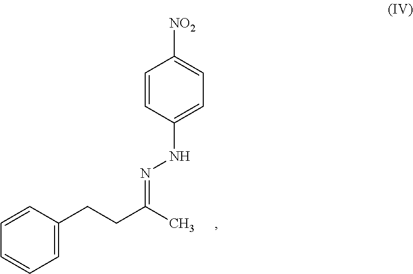 [SMILES: C/C(CCc1ccccc1)=N\Nc1ccc([N+](=O)[O-])cc1]
for use in modulating cytokinesis to treat a disease condition in a subject.

14. The compound of claim 13, wherein the compound is administered by systemic delivery selected from the group consisting of oral, parenteral, intranasal, sublingual, rectal, and transdermal administration.

15. A pharmaceutical composition for modulating cell mechanics of a disease condition in a subject comprising a compound having the formula: 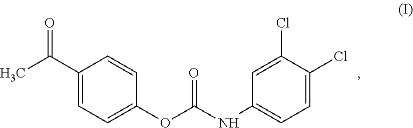 [SMILES: CC(=O)c1ccc(OC(=O)Nc2ccc(Cl)c(Cl)c2)cc1]
wherein the pharmaceutical composition further comprises at least one pharmaceutically-acceptable carrier.

16. A pharmaceutical composition for modulating cell mechanics of a disease condition in a subject comprising a compound having the formula: 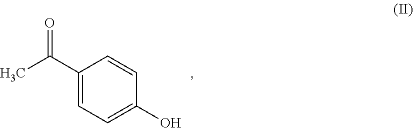
wherein the pharmaceutical composition further comprises at least one pharmaceutically-acceptable carrier.

17. The pharmaceutical composition of claim 16, and the composition further comprises a compound having the formula:

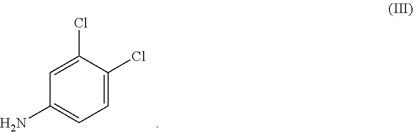

18. A pharmaceutical composition for modulating cytokinesis of a disease condition in a subject comprising a compound having the formula: 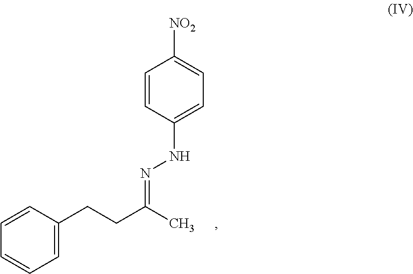 [SMILES: C/C(CCc1ccccc1)=N\Nc1ccc([N+](=O)[O-])cc1]
wherein the pharmaceutical composition further comprises at least one pharmaceutically-acceptable carrier.

19. An in vivo, large-scale and high-throughput screening method for identifying cell mechanical modulator, the screening method comprising the steps of:
(a) obtaining cells and placing the cells on multiple-well substrate plates for cytokinesis;
(b) contacting the cells on multiple-well substrate plates with compound candidates;
and (c) monitoring and analyzing the cytokinesis and the growth of the cells.

20. The screening method of claim 19, wherein the cells are from Dictyostelium discoideum strains.

21. The screening method of claim 20, wherein Dictyostelium discoideum strains comprise wild type and mutants.

22. A method for identifying compounds as cell mechanical modulators using the screening method of claim 19.
